(19) United States
(12) Patent Application Publication
(10) Pub. No.: US 2024/0254247 A1
(43) Pub. Date: Aug. 1, 2024

(54) Anti-BAFF Antibodies for use in a Method of Treatment of Long Covid and/or Post-Acute Sequelae SARS-CoV-2 Infection (PASC)

(71) Applicants: GlaxoSmithKline Intellectual Property Limited, Stevenage (GB); GlaxoSmithKline Intellectual Property Management Limited, Stevenage (GB)

(72) Inventors: Robert Brian HENDERSON, Stevenage (GB); Jeremy SOKOLOVE, Collegeville, PA (US); André VAN MAURIK, Stevenage (GB)

(73) Assignees: GlaxoSmithKline Intellectual Property Limited, Stevenage (GB); GlaxoSmithKline Intellectual Property Management Limited, Stevenage (GB)

(21) Appl. No.: 18/566,136

(22) PCT Filed: Jun. 15, 2022
(86) PCT No.: PCT/EP2022/066381

§ 371 Date: Dec. 1, 2023

Related U.S. Application Data
(60) Provisional application No. 63/211,686, filed on Jun. 17, 2021.

(51) Int. Cl. C07K 16/28; A61K 39/00

(52) U.S. Cl. CPC C07K 16/2875; A61K 2039/505

(57) ABSTRACT

The present disclosure relates to a B lymphocyte Stimulator (BlyS; B-cell activating factor; BAFF) antagonist for use in the treatment of Long Covid and/or post-acute sequelae SARS-COV-2 infection (PASC). Also disclosed is a BlyS antagonist for use in the treatment of an autoimmune condition induced following a viral infection. Such autoimmune conditions may be chronic, such as Long Covid. Also provided is a method for the treatment of an autoimmune condition induced following a viral infection comprising administering to a subject in need thereof a therapeutically effective amount of a BlyS antagonist.

### FIELD OF THE INVENTION

The present invention relates to a BlyS antagonist, such as an anti-BlyS antibody for example the antibody belimumab, for use in the treatment of Long Covid and/or post-acute sequelae SARS-COV-2 infection (PASC).

Also provided is a BlyS antagonist, such as an anti-BlyS antibody, for use in the treatment of an autoimmune condition induced following a virus infection, such as human coronavirus SARS-COV-2 or COVID-19. Such autoimmune conditions may be chronic, such as Long Covid and/or post-acute sequelae SARS-COV-2 infection (PASC). Also provided is a method of treatment for an autoimmune condition induced following a virus infection, such as Long Covid and/or post-acute sequelae SARS-COV-2 infection (PASC), in a subject in need thereof, said method comprising administering to said subject a therapeutically effective amount of a BlyS antagonist, such as an anti-BlyS antibody.

Also provided is the use of a BlyS antagonist in the manufacture of a medicament for the treatment of an autoimmune condition induced following a virus infection, such as COVID-19, and a pharmaceutical composition comprising a BlyS antagonist for use in the treatment of an autoimmune condition induced following a viral infection, such as COVID-19.

### BACKGROUND TO THE INVENTION

COVID-19 was declared a Public Health Emergency of International Concern on 30 Jan. 2020, following its emergence in China in November 2019. At the time of writing, over 168 million cases have been confirmed and over 3.4 million deaths have been reported globally.

The infectious agent has been identified as a coronavirus (known as severe acute respiratory syndrome coronavirus-2, SARS-COV-2 and previously known as 2019-nCOV2) capable of spreading by human to human transmission. Other coronaviruses that are pathogenic to humans are associated with mild clinical symptoms, with two notable exceptions: severe acute respiratory syndrome (SARS) coronavirus (SARS-COV) and Middle East respiratory syndrome (MERS) coronavirus (MERS-COV).

Coronaviruses consist of an enveloped single strand positive sense RNA genome of 26 to 32 kb in length. Coronaviruses utilise membrane bound spike proteins to bind to a host cell surface receptor to gain cellular entry. Following entry into the host cell, the RNA genome is translated into two large polypeptides by the host ribosomal machinery. The polypeptides are processed by two proteases, the coronavirus main proteinase (3CL-Pro) and the papain-like proteinase to generate the proteins required for viral replication and packaging.

Coronaviruses are classified by phylogenetic similarity into four categories: α (e.g. 229E and NL-63), β (e.g. SARS-COV-2, SARS-COV, MERS-COV and OC43), γ and δ. SARS-COV-2 has been reported to have 79% sequence identity to SARS-COV, and certain regions of the SARS-COV-2 genome exhibit greater or lesser degrees of conservation to SARS-COV.

The spike protein of SARS-COV and SARS-COV-2 (and the a coronavirus NL63) share the same host cell receptor, namely angiotensin converting enzyme 2 (ACE2). This is highly expressed in, inter alia, type II alveolar cells, and in the epithelial cells from the oral cavity (particularly the tongue). The nature of binding, however, would appear to be distinct. As evidence of this, the S ectodomain of SARS-COV-2 would appear to bind to ACE2 with 10- to 20-fold higher affinity compared to the S protein of SARS-COV. In addition, whilst three monoclonal antibodies raised against the receptor binding domain of SARS-COV demonstrated strong binding to the receptor binding domain of SARS-COV at 1 μM, no binding to the receptor binding domain of SARS-COV-2 could be detected at this concentration. This likely reflects differences at the sequence level. The receptor binding domain of SARS-COV-2 and SARS-COV have an overall sequence identity of 73.5%. Further, a number of residues of the SARS-COV receptor known to be involved in binding to ACE2 are not conserved in SARS-COV-2.

The genome of SARS-COV-2 has been sequenced in vast numbers of patients worldwide. To date, GISAID (Global Initiative on Sharing All Influenza Data) have identified eight global strains (S, O, L, V, G, GH, GR and GV). Two early strains were designated L (original strain detected in Wuhan December 2019) and S (the first mutant strain) with one amino acid change observed in ORF8 at position 84 (serine in strain S and leucine in strain L). Other variants of the virus include the British Alpha variant (VUI-202012/01, strain GR, lineage B.1.1.7), the South African Beta variant (20H/501Y.V2, strain GH, lineage B.1.351), the Brazilian Gamma variant (Lineage P.1 or B.1.1.28.1, strain GR), the Indian Delta variant (lineage B.1.617.2, strain G/452R.V3) and the Omicron variant (strain GR/484A, lineage B.1.1.529). VUI-202012/01 is defined by a set of 17 mutations, two of the most significant are N501Y and E484K mutations in the spike protein. The South African variant contains 3 key amino acid mutations in the receptor binding domain of the spike protein: N501Y, K417N and E484K. Lineage P.1 has 10 mutations in its spike protein, including N501Y and E484K. Lineage B.1.617.2 has been designated a so called “double mutant” with E484Q and L452R mutations in its spike protein. The N501Y mutation is within the receptor binding domain of the spike protein, the part which binds to the human ACE2 receptor (the receptor the virus uses to enter host cells). Therefore, changes in this part of spike protein may likely result in the virus becoming more infectious and enhancing transmissibility between people. It is understood that further variants and subvariants will emerge over time.

Naturally the largest focus on the treatment of COVID-19 has been on the acute stages of the disease, however over time the impact of a disease termed “long-Covid” or post-acute sequelae SARS-COV-2 infection (PASC) has emerged. This prolonged illness is often seen in populations of patients who had severe COVID-19 requiring hospitalisation, especially among older adults, but also more recently is being seen in an increasing number of SARS-COV-2 infections in persons first evaluated as outpatients, including younger adults with milder disease.

Currently available therapies have limited clinical benefit in the more severe or acute stages of hospitalised COVID-19 when patients require high-flow oxygen or invasive mechanical ventilation. To date, no targeted therapy has been proven to have sufficient benefit in improving recovery from COVID-19 or long-Covid or post-acute sequelae SARS-COV-2 infection (PASC).

It is imperative that medicines are found which can treat these diseases, minimise the time patients require medical intervention and relieve some of the pressure on global health services and restore quality of life to patients post infection.

### SUMMARY OF THE INVENTION

Therefore, according to a first aspect of the invention, there is provided a BlyS antagonist for use in the treatment of Long Covid and/or post-acute sequelae SARS-COV-2 infection (PASC). In a second aspect, there is provided a Blys antagonist for use in the treatment of an autoimmune condition induced following a viral infection. In another aspect, there is provided use of a BlyS antagonist in the manufacture of a medicament for the treatment of Long Covid and/or post-acute sequelae SARS-COV-2 infection (PASC).

In another aspect, there is provided use of a BlyS antagonist in the manufacture of a medicament for the treatment of an autoimmune condition induced following a virus infection. In a further aspect, there is provided a pharmaceutical composition for use in the treatment of of Long Covid and/or post-acute sequelae SARS-COV-2 infection (PASC), said pharmaceutical composition comprising a BlyS antagonist.

According to a yet further aspect, there is provided a method for the treatment of Long Covid and/or post-acute sequelae SARS-COV-2 infection (PASC) in a subject in need thereof comprising administering to said subject a therapeutically effective amount of a Blys antagonist.

In a further aspect, there is provided a method for the treatment of Long Covid and/or post-acute sequelae SARS-COV-2 infection (PASC) in a subject in need thereof, said method comprising administering to said subject a therapeutically effective amount of an anti-BlyS antibody, for example wherein the anti-BlyS antibody is belimumab and/or is as defined herein. In a further aspect there is provided a method for the treatment of Long Covid and/or post-acute sequelae SARS-COV-2 infection (PASC) comprising taking a sample from a patient, testing for the presence of at least one or more of serum cytokines, Blys, IFN-β, PTX3, IFN-lambda 2/3 and/or IL-6 and comparing the levels to that of a healthy reference wherein if the level of the cytokines tested is higher than that of the reference level (such as 2-fold higher BLyS levels or 6-fold higher IFN levels or 2-fold higher IFN-lambda levels) then treating the patient with a Blys antagonist such as an anti-Blys antibody. Thus, in one embodiment the anti-BlyS antibody or combinations as defined herein are administered to a subject after testing for the presence of serum cytokines, Blys, IFN-β, PTX3, IFN-lambda 2/3 and/or IL-6. 13.

### BRIEF DESCRIPTION OF FIGURES

FIG. 1—Volcano plot of normalized protein expression (NPX) differences between the CR and PASC cohort. Targets with an adj. p value<=0.05 highlighted. BLyS levels circled. Annotations of BLyS and other proteins of interest provided.

FIG. 2—Normalized BLyS levels for each patient from indicated group. (FIG. 2a) Comparison of BLyS values in recovery groups compared to pre-pandemic healthy controls and uninfected patients with active SLE. Horizontal bars—mean. (FIG. 2b) Direct comparison of recovery cohorts with BLyS positivity cutoff displayed. Percentages indicate percent of patients exceeding the BLyS positivity cutoff.

FIG. 3—BLyS correlations with biomarkers of COVID-19. (FIG. 3a) Correlation of normalized BLyS levels with normalized CXCL10 levels in the plasma of CR and PASC patient groups. (FIG. 3b) Correlation of normalized BLyS levels with normalized Pentraxin 3 (PTX3) levels in the plasma of CR and PASC patient groups.

FIG. 4—Flow cytometry assessment of B cell profiles in high versus low BLyS PASC patients. (FIG. 4a) Antibody secreting cell frequency of total CD19+ cells in BLyS− vs. BLyS+ PASC patients. (FIG. 4b) Activated naive cell frequency of total CD19+ cells in BLyS− vs. BLyS+ PASC patients. (FIG. 4c) Double negative 2 cell frequency of total CD19+ cells in BLyS− vs. BLyS+ PASC patients. (FIG. 4d) Log 2-transformed ratio of Double negative 2 cells to Double negative 1 cells as a metric of EF:GC B cell activity.

FIG. 5—Autoreactivity in in the PASC cohort was screened by Exagen Inc for reactivity against 31 clinically-relevant autoantigens. Heatmap of patient results. Each column represents a single patient grouped by the total number of autoreactive positive tests that the patient displayed. Bolded boxes represent clinical positive tests with the depth of color indicating the magnitude of the test result. Scale for each test is documented below the heatmap.

FIG. 6—Contingency testing of PASC patient symptoms in BLyS− versus BLyS+ patients. P value represents Fisher's Exact contingency testing.

### DETAILED DESCRIPTION OF THE INVENTION

In the process of investigating the immunological underpinnings of the disease, a non-canonical B cell activation pathway, the extrafollicular (EF) pathway, has been identified as a significant component of the immune response in severe disease (Woodruff et al., Nat Immunol 21, 1506-1516 (2020); https://doi.org/10.1038/s41590-020-00814-z). This pathway has been previously identified in active and flaring autoimmune disease such as systemic lupus erythematosus (SLE), implicated in the development of autoreactive responses, and correlated with severe disease outcomes. Circulating B cells in critically ill patients with COVID-19 are phenotypically similar to the extrafollicular B cells that were previously identified in patients with autoimmune diseases such as SLE. Interestingly, the frequency of extrafollicular B cells in patients with COVID-19 correlated with the early production of high titres of neutralising antibodies as well as with inflammatory biomarkers (such as C-reactive protein) and organ damage. Consistent with Woodruff et al., Kaneko et al. (Kaneko, N. et al., Cell 183, 143-157.e13 (2020); https://doi.org/10.1016/j.cell.2020.08.025) also reported increased levels of extrafollicular IgD− CD27− B cells in COVID-19 post-mortem lymph node and spleen samples. IgD− CD27− “double-negative” B cells are considered to be “disease-related” cells and are generally described as “extra-follicular”, implying they are not derived from the germinal center reaction but are frequently class switched and have the hallmarks of having been induced in a T-dependent manner without germinal-center-based selection. Data indicate that, in the absence of germinal center formation in COVID-19, an extra-follicular type of class-switched B cell response, more typical of disease rather than of long-lasting protection, predominates in secondary lymphoid organs. Patients with severe/critical COVID-19 have now been identified displaying clinical autoreactivities such as anti-nuclear antibodies (ANAs), autoantibodies against phospholipids, type-I interferons, rheumatoid factor (RF), and other self antigens. The presence of autoreactivity could be correlated with increasing serum levels of CRP; confirming autoreactivity as a common feature of severe disease. Of importance, all patients displaying additional autoreactivities tested positive for either ANAs or RF, suggesting that these two clinical tests may be valuable in efficiently screening patients for broad tolerance breaks. Using Rapid Extracellular Antigen Profiling (REAP), Wang et al. (Wang E Y, et al., preprint at. medRxiv (2020); https://doi.org/10.1101/2020.12.10.20247205) identified a wide range of autoantigens targeted by antibodies in patients with severe COVID-19. These include antibodies against immunomodulatory proteins such as cytokines, interferons, chemokines and leukocytes, which could directly affect the course of antiviral immunity by antagonising innate antiviral responses, thus impacting disease progression as well as antibodies to tissue-specific antigens expressed in the central nervous system, vasculature, connective tissues, cardiac tissue, hepatic tissue and intestinal tract, which could potentially cause antibody-mediated organ damage. Importantly, autoantibodies targeting tissue-associated antigens were shown to correlate with disease severity and clinical characteristics in COVID-19 patients. Longitudinal REAP analysis revealed the existence of both pre-existing autoantibodies, as well as a broad subset of de novo autoantibodies that were induced following infection.

Moreover, autoantibodies have been found in people with long-term symptoms of COVID-19 (Long Covid) and/or post-acute sequelae SARS-COV-2 infection (PASC) months after infection. Type I interferon induction and signalling have key roles in preventing lethal COVID-19. It has been described that neutralising antibodies to type I interferons predispose patients to life-threatening COVID-19. In one study 135 of 987 (13.7%) patients with severe COVID-19 had antibodies to IFNα, IFNω or both, a finding that was later confirmed by another study. By contrast, none of the 663 patients with asymptomatic or mild COVID-19 and only 4 of the 1,227 (0.3%) healthy donors had autoantibodies to type I interferon. Collectively, these studies not only show the devastating consequences of lack of type I interferons in COVID-19 but also the significance of autoantibodies in impacting the disease course. As a result, and with the identification of an autoantibody component in multisystemic inflammatory syndrome in children (MIS-C), it is reasonable to consider the influence of these systems on antiviral immunity and on the observed clinical phenomena of long-term post-COVID-19 sequelae emerging in large numbers of recovered patients. Increased BLyS levels have been shown to promote the survival and activation of autoreactive B cells even in the absence of T cells. Although these observations support the notion that SARS-COV-2 infection frequently results in breaks of self-tolerance to a variety of autoantigens, the exact contribution of Blys levels and BLyS-driven breaks of self-tolerance to 1) severe COVID-19 infection through the generation of antibodies against immunomodulatory proteins and 2) post COVID-19 symptoms through the generation of pathogenic autoantibodies remains to be demonstrated.

Antiphospholipid (aPL) antibodies as a class have been reported at the highest frequency of all autoantibodies, detected in about half of severe cases [Zuo Sci Transl Med. 2020; 12(570)] and being highest in those in the Intensive Care Unit (ICU), affecting up to 91% of COVID-19 patients with a prolonged activated partial thromboplastin time (aPTT) [Bowles New Eng J Med. 2020; 383(3):288-90] It was found that the presence of neutrophil extracellular traps (NETs), which are prothrombotic in antiphospholipid syndrome (APS), were associated with higher titers of aPL antibodies in patients with COVID-19. IgG fractions purified from patients with severe COVID-19 were furthermore shown to accelerate thrombosis when injected into mice, as demonstrated in other studies of APS. These findings indicate a potential role of aPL antibodies in potentiating thrombosis in hospitalized patients with COVID-19 through promotion of NET formation.

It is critical to understand the immunological environment resulting in the activation of these pathways, understand if they are related to the de novo formation of new autoreactivity and emergence of post-COVID-19 symptoms, and identify potential therapeutic targets for treatment of both acute and long-term symptoms resulting from COVID-19.

### Definitions

The term “BlyS antagonist” as used herein refers to an agent which reduces or blocks BlyS activity, for example by binding to BlyS or the BR3, TACI or BCMA receptors. In one embodiment the BlyS antagonist is a small chemical molecule. In another embodiment, the BlyS antagonist is an anti-BlyS binding protein or an anti-BlyS receptor binding protein. In one embodiment the BlyS antagonist binds specifically to BlyS. In another embodiment the BlyS antagonist is an anti-BlyS antibody. In one embodiment the anti-Blys antibody is belimumab or a variant thereof.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanised, multispecific antibodies, including bispecific antibodies, and heteroconjugate antibodies; antigen binding antibody fragments, Fab, F(ab′)₂, Fv, disulphide linked Fv, single chain Fv, disulphide-linked scFv, diabodies, TANDABS, etc. and modified versions of any of the foregoing (for a summary of alternative “antibody” formats see Holliger and Hudson, Nature Biotechnology, 2005, Vol 23, No. 9, 1126-1136).

The term “anti-BlyS antibody” as used herein refers to antibodies, which are capable of binding to BlyS and affecting its function, e.g. reducing and/or blocking in the case of an antagonist antibody as described herein. The term “antibody” is used in the broadest sense as defined hereinbefore and specifically covers monoclonal antibodies, polyclonal antibodies, multispecific antibodies and antibody fragments exhibiting the desired biological activity.

Anti-BlyS antibodies of the invention are those which are capable of antagonising BlyS and may decrease, block or inhibit BlyS-induced signal transduction. For example, anti-BlyS binding antibodies of the invention may disrupt and/or block the interaction between BlyS and its receptor to inhibit or downregulate BlyS-induced signal transduction. In particular, anti-BlyS binding antibodies of the invention which prevent BlyS induced signal transduction by specifically recognising the unbound BlyS protein, receptor-bound BlyS protein, or both unbound and receptor-bound BlyS protein can be used in accordance with the invention described herein. The ability of an anti-BlyS antibody of the invention to inhibit or downregulate BlyS induced signal transduction may be determined by techniques known in the art. For example, BlyS-induced receptor activation and the activation of signalling molecules can be determined by detecting the phosphorylation (e.g. tyrosine or serine/threonine) of the receptor or a signaling molecule by immunoprecipitation followed by western blot analysis. In one embodiment, the anti-BlyS antibody is a monoclonal antibody. In another embodiment, the anti-BlyS antibody is a human or humanized antibody.

In one embodiment, the anti-BlyS antibody comprises at least one or more of CDRH1 of SEQ ID NO: 1; CDRH2 of SEQ ID NO: 2; CDRH3 of SEQ ID NO: 3; CDRL1 of SEQ ID NO: 4; CDRL2 of SEQ ID NO: 5; or CDRL3 of SEQ ID NO: 6. In one embodiment, the anti-BlyS antibody comprises CDRH1 of SEQ ID NO: 1; CDRH2 of SEQ ID NO: 2; CDRH3 of SEQ ID NO: 3; CDRL1 of SEQ ID NO: 4; CDRL2 of SEQ ID NO: 5; and CDRL3 of SEQ ID NO: 6. In a further embodiment, the anti-BlyS antibody comprises at least three or more of CDRH1 of SEQ ID NO: 1; CDRH2 of SEQ ID NO: 2; CDRH3 of SEQ ID NO: 3; CDRL1 of SEQ ID NO: 4; CDRL2 of SEQ ID NO: 5; or CDRL3 of SEQ ID NO: 6. In another embodiment, the anti-BlyS antibody comprises CDR sequences which are at least 95%, or 96% or 97% or 98% or 99% homologus to CDRH1 of SEQ ID NO: 1; CDRH2 of SEQ ID NO: 2; CDRH3 of SEQ ID NO: 3; CDRL1 of SEQ ID NO: 4; CDRL2 of SEQ ID NO: 5; or CDRL3 of SEQ ID NO: 6.

In another embodiment, the anti-BlyS antibody comprises CDR sequences which are variant CDR sequences according to CDRH1 of SEQ ID NO: 1; CDRH2 of SEQ ID NO: 2; CDRH3 of SEQ ID NO: 3; CDRL1 of SEQ ID NO: 4; CDRL2 of SEQ ID NO: 5; or CDRL3 of SEQ ID NO: 6 and wherein a variant has no more than two or no more than one amino acid changes in each CDR.

In one embodiment, the anti-Blys antibody comprises all six of CDRH1 of SEQ ID NO: 1; CDRH2 of SEQ ID NO: 2; CDRH3 of SEQ ID NO: 3; CDRL1 of SEQ ID NO: 4; CDRL2 of SEQ ID NO: 5; and CDRL3 of SEQ ID NO: 6

In a yet further embodiment, the anti-BlyS antibody comprises a variable heavy chain sequence with at least 95% or 97% or 98% or 99% homology to SEQ ID NO: 7 and a light chain variable sequence with at least 95% or 97% or 98% or 99% homology to of SEQ ID NO: 8.

In a yet further embodiment, the anti-BlyS antibody comprises at least one of a variable heavy chain sequence of SEQ ID NO: 7 and a light chain variable sequence of SEQ ID NO: 8. In another embodiment, the anti-BlyS antibody comprises both of variable heavy chain sequence of SEQ ID NO: 7 and light chain variable sequence of SEQ ID NO: 8. In a further embodiment, the anti-BlyS antibody comprises a heavy chain sequence of SEQ ID NO: 9 and a light chain sequence of SEQ ID NO: 10. In a yet further embodiment, the anti-BlyS antibody consists of a heavy chain sequence of SEQ ID NO: 9 and a light chain sequence of SEQ ID NO: 10. In a still further embodiment the anti-BlyS antibody is belimumab or variants thereof. In a still further embodiment the anti-Blys antibody binds to the same epitope as belimumab.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRS. Similarly, “CDRH” refers to a heavy chain CDR, such as all three heavy chain CDRs, and “CDRL” refers to a light chain CDR, such as all three light chain CDRS.

Throughout this specification, amino acid residues in variable domain sequences and variable domain regions within full-length antigen binding sequences, e.g. within an antibody heavy chain sequence or antibody light chain sequence, are numbered according to the Kabat numbering convention. Similarly, the terms “CDR”, “CDRH1”, “CDRH2”, “CDRH3”, “CDRL1”, “CDRL2”, “CDRL3” used to define the antagonist anti-BlyS binding proteins described herein, follow the Kabat numbering convention or HuCAL (Human Combinatorial Antibody Library) numbering system. For further information, see Kabat et al., Sequences of Proteins of Immunological Interest, 4th Ed., U.S. Department of Health and Human Services, National Institutes of Health (1987).

It will be apparent to those skilled in the art that there are alternative numbering conventions for amino acid residues in variable domain sequences and full-length antibody sequences. There are also alternative numbering conventions for CDR sequences, for example those set out in Chothia et al. (1989) Nature 342: 877-883. The structure and protein folding of the antigen binding protein may mean that other residues are considered part of the CDR sequence and would be understood to be so by a skilled person. Yet other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

Table A below represents one definition using each numbering convention for each CDR or binding unit. The Kabat numbering scheme is used in Table A to number the variable domain amino acid sequence. It should be noted that some of the CDR definitions may vary depending on the individual publication used.

TABLE A

| | | | | | Minimum |
|---|---|---|---|---|---|
| | Kabat | Chothia | AbM | Contact | binding |
| | CDR | CDR | CDR | CDR | unit |
| H1 | 31-35/ | 26-32/ | 26-35/ | 30-35/ | 31-32 |
| | 35A/35B | 33/34 | 35A/35B | 35A/35B | |
| H2 | 50-65 | 52-56 | 50-58 | 47-58 | 52-56 |
| H3 | 95-102 | 95-102 | 95-102 | 93-101 | 95-101 |
| L1 | 24-34 | 24-34 | 24-34 | 30-36 | 30-34 |
| L2 | 50-56 | 50-56 | 50-56 | 46-55 | 50-55 |
| L3 | 89-97 | 89-97 | 89-97 | 89-96 | 89-96 |

### Pharmaceutical Compositions, Routes of Administration & Dosages

The BlyS antagonist, such as the anti-BlyS antibody (e.g. belimumab) as described herein can be administered by various routes of administration, typically parenteral. This is intended to include intravenous, intramuscular, subcutaneous, rectal and vaginal. Effective dosages will depend on the condition of the patient, age, weight, or any other treatments, among other factors. The administration may be effected by various protocols, e.g. weekly, bi-weekly, or monthly, dependent on the dosage administered and patient response.

In one embodiment, the anti-BlyS antibody (e.g. belimumab) is administered intravenously (IV), such as by intravenous injection. Depending on the type and severity of the disease, about 1 μg/kg to 50 mg/kg of bodyweight, or more specifically between about 0.1 mg/kg to 20 mg/kg of bodyweight, of belimumab is a candidate initial dosage for administration to a subject, whether, for example, by one or more separate administrations, or by continuous infusion. More specifically the dosage of the antibody will be in the range from about 0.05 mg/kg of bodyweight to about 10 mg/kg of bodyweight. In one embodiment when the anti-BlyS antibody (e.g. belimumab) is given intravenously the recommended dosage regimen is 10 mg/kg. Thus, in one embodiment the anti-BlyS antibody is administered to a subject at a dose of 10 mg/kg.

In one embodiment, the anti-BlyS antibody is administered once per week. In another embodiment, the anti-BlyS antibody (e.g. belimumab) is administered every 2 weeks. Thus, in one embodiment, the anti-BlyS antibody is administered at 10 mg/kg every 2 weeks. In a further embodiment, the anti-BlyS antibody is administered at 10 mg/kg at 2 week intervals for the first 3 doses and at 4-week intervals thereafter. In other embodiments, the anti-BlyS antibody is administered every week, every 2 weeks or every 3 weeks. In one embodiment, the anti-BlyS antibody is administered every 2 weeks, which means that the anti-BlyS antibody is administered at 2 week intervals, for example 3 doses in 4 weeks at day 0, day 14 and day 28. In a further embodiment, the anti-BlyS antibody is administered every 2 weeks (i.e. following administration at day 0) for at least 4 weeks, for at least 6 weeks or for at least 8 weeks, and then every 4 weeks thereafter.

In one embodiment, the anti-BlyS antibody (e.g. belimumab) is administered subcutaneously, such as by a subcutaneous injection. In one such embodiment, the anti-BlyS antibody is administered to a subject at a unit dose of 200 mg.

Subcutaneous injections of the present invention may be administered as single injections wherein the entire dose is administered as a single shot, wherein the entire volume of the dose is administered all at once. A single shot injection may be administered multiple times. A single shot differs from a continuous or titrated administration, e.g. an infusion, wherein the administration may be administered over several minutes, hours or days until a full dose is achieved.

In one embodiment, the anti-BlyS antibody is administered to a subject at a unit dose of 200 mg. In a further embodiment, the anti-BlyS antibody is administered once every week. In a further embodiment, the anti-BlyS antibody is administered once per week at a unit dose of 200 mg, i.e. at a unit dose of 200 mg a week. In another embodiment, the anti-BlyS antibody is administered twice a week at predominantly the same time point, for example within the same hour or for example the same day. In an alternative embodiment, the anti-BlyS antibody (e.g. belimimab) is administered at a total dose of 400 mg. Thus, in a further embodiment the anti-BlyS antibody (e.g. belimumab) is administered to a subject at a unit dose of 400 mg a week. In one embodiment, the 400 mg dose may be provided by more than one injection, for example a 200 mg unit dose is administered twice. The anti-BlyS antibody may be administered at the same or different injection sites but is preferably administered at different injection sites. In a further embodiment, the anti-BlyS antibody is administered twice weekly, sequentially or concomitantly, at different reaction sites. In yet a further embodiment, the anti-BlyS antibody is administered at a dose of 400 mg a week for 4 weeks, for example at day 0, day 7, day 14, day 21 and day 28, and then at a dose of 200 mg once weekly thereafter. In another embodiment the anti-BlyS antibody is administered at a dose of 400 mg a week (i.e. following administration at day 0) for at least 4 weeks, or for at least 8 weeks or for at least 12 weeks, and then at a dose of 200 mg once weekly thereafter.

In further embodiments, the anti-BlyS antibody (e.g. belimumab) is administered intravenously (IV) prior to subcutaneous administration. According to such embodiments, the initial IV dose is a so called “loading dose” prior to subcutaneous administration. The loading dose may be about 1 μg/kg to 50 mg/kg of bodyweight, or more specifically between about 0.1 mg/kg to 20 mg/kg of bodyweight, of anti-BlyS antibody. Specifically the loading dose of the antibody may be in the range from about 0.05 mg/kg of bodyweight to about 10 mg/kg of bodyweight. In one embodiment, the antibody is administered intravenously at a loading dose of 10 mg/kg for at least 1 week prior to subcutaneous administration.

In further embodiments, the BlyS antagonist, such as the anti-BlyS antibody, is administered in combination with a further therapeutic agent. Thus, in one embodiment the treatment further comprises administration of a further therapeutic agent. Such further therapeutic agents will be appreciated by and apparent to the skilled person in the context of the disease to be treated, treatment to be conducted and/or needs of the subject in need thereof. For example in some embodiments, the additional therapeutic agent is an anti-viral and/or antibiotic agent. In yet further embodiments, the additional therapeutic agent is a steroid, corticosteroid or antimalarial agent.

In one embodiment, the further therapeutic agent is a CD20 antagonist. Thus, in a particular embodiment, the BlyS antagonist (e.g. the anti-BlyS antibody) is administered in combination with a CD20 antagonist. In a further embodiment, the CD20 antagonist is a CD20 binding protein. In a yet further embodiment the CD20 antagonist is an anti-CD20 antibody. For example in one embodiment, the anti-CD20 antibody is rituximab.

Rituximab is a chimeric gamma 1 anti-human CD20 antibody. The complete amino acid and corresponding nucleic acid sequence for this antibody may be found in U.S. Pat. No. 5,736,137. Rituximab may be administered by various routes of administration, typically parenteral. This is intended to include intravenous, intramuscular, subcutaneous, rectal and vaginal. Effective dosages will depend on the condition of the patient, age, weight, or any other treatments, among other factors. The administration may be effected by various protocols, e.g., weekly, bi-weekly, or monthly, dependent on the dosage administered and patient response.

In one embodiment, rituximab is administered as an intravenous infusion.

In another embodiment, rituximab is administered at a dosage of 1000 mg.

Other dosages at which rituximab may be administered include a dosage of 500 mg; a dosage of 375 mg/m² IV once weekly for 4 doses at 6 month intervals to a maximum of 16 doses; a dosage of 375 mg/m² IV every 8 weeks for 12 doses and a dosage of 375 mg/m² IV once weekly for 4 doses.

In one embodiment, rituximab is administered as a subcutaneous injection. In one such embodiment the rituximab is at a concentration of 120 mg/ml. In yet a further embodiment patients who receive a subcutaneous administration must first have received an intravenous dose. In another embodiment, rituximab is administered at a dosage of 1400 mg.

In one embodiment, the anti-CD20 binding antibody which is capable of depleting B cells is ofatumumab.

Ofatumumab is a human monoclonal anti-human CD20 antibody. The complete amino acid and corresponding nucleic acid sequence for this antibody may be found in U.S. Pat. No. 8,529,902.

Ofatumumab may be administered by various routes of administration, typically parenteral. This is intended to include intravenous, intramuscular, subcutaneous, rectal and vaginal. Effective dosages will depend on the condition of the patient, age, weight, or any other treatments, among other factors.

As an example, ofatumumab may be administered as an intravenous infusion, at a dosage of 1000 mg. Ofatumumab may also be administered at an initial dosage of 300 mg, followed by 1,000 mg on Day 8 (Cycle 1). Ofatumumab may also be administered at an initial dosage of 2000 mg weekly for 7 doses, followed 4 weeks later by 2,000 mg every 4 weeks for 4 doses. As established earlier, any other CD20 binding antibodies capable of depleting B cells will be equally suitable at similar dosing regimens schedules in the context of the invention.

The anti-BlyS antibody and the further therapeutic agent, such as the anti-CD20 antibody, may be administered simultaneously, concurrently or sequentially. For example, the anti-BlyS antibody may be given before the anti-CD20 antibody or after the anti-CD20 antibody. Thus, in one embodiment the anti-CD20 antibody is administered to the subject in need thereof after the anti-BlyS antibody.

In a further embodiment the anti-CD20 antibody is administered at least two weeks after the first dose of the anti-BLyS antibody. In another embodiment, the anti-CD20 binding antibody is administered at least twice between weeks 2 and 20 after the first dose of the anti-BLyS antibody. For example, the anti-CD20 binding antibody may be administered at least at weeks 2 and 20, weeks 4 and 18, weeks 6 and 16, weeks 8 and 14 or weeks 10 and 12 after the first dose of the anti-BlyS antibody. In some embodiments, the anti-CD20 binding antibody is administered at weeks 4 and 6, weeks 6 and 10, weeks 8 and 10 or weeks 8 and 12 after the first dose of the anti-BlyS antibody. In a further embodiment, the anti-CD20 binding antibody is administered 8 and 10 weeks after the first dose of the anti-BLyS antibody. In a yet further embodiment, the anti-CD20 binding antibody is administered 4 and 6 weeks after the first dose of the anti-BlyS antibody.

Additional doses of the anti-CD20 binding antibody may be administered at least 24 weeks after the start of the treatment with the anti-BlyS antibody. For example, third and fourth doses of the anti-CD20 binding antibody which is capable of depleting B cells may be administered at weeks 24 and 48, or weeks 24 and 25, 26, 27, 28, 29, 30, 31, 32, 33, 34, 35, 36, 37, 38, 39, 40, 41, 42, 43, 44, 45, 46, 47 or 48. Similarly, a fourth dose may be administered at least 48 weeks after the start of the treatment with the anti-BlyS antibody. For example, a fourth dose may be administered at week 48, 49, 50, 51, 52, 53, 54, 55, 56, 57, 58, 59, 60, 61, 62, 63, 64, 65, 66, 67, 68, 69, 70, 71 or 72.

In one embodiment, the anti-CD20 antibody and the BlyS antagonist, such as an anti-BlyS antibody, is provided as a combination for use in the treatment of Long Covid and/or post-acute sequelae SARS-COV-2 infection (PASC) or alternatively for use in the treatment of an autoimmune condition induced following a viral infection. In a further embodiment, the anti-BlyS antibody belimumab and the anti-CD20 antibody rituximab is provided as a combination for use in the treatment of Long Covid and/or post-acute sequelae SARS-COV-2 infection (PASC) or alternatively for use in the treatment of an autoimmune condition induced following a viral infection. In a yet further embodiment, the autoimmune condition induced following a viral infection is as defined herein and the combination of anti-BlyS antibody and anti-CD20 antibody is provided for use in the treatment of said condition.

In one embodiment, anti-BlyS antibody is administered for a period of 24 weeks. In another embodiment, anti-BlyS antibody is administered for a period of 52 weeks.

Administration of the BlyS antagonist (e.g. an anti-BlyS antibody) and/or the combinations defined herein for use in the treatment of Long Covid and/or PASC or in an autoimmune condition induced following a viral infection, increases the immunological tolerance of the subject and/or induces a long term remission of said condition. Such increase in immunological tolerance and/or induction of long term remission can be measured by clinical or biomarker assessment or by use of a suitable disease severity score. In another embodiment the anti-BlyS antibody or combination as defined herein are administered to a subject for a period until the immunological tolerance and/or remission is induced in the subject. Such administration may include wherein the anti-BlyS antibody is administered for a continued period after administration of the combination has ceased or after administration of the anti-CD20 antibody has ceased. Alternatively, the anti-CD20 antibody may be administered for a continued period after administration of the combination has ceased or after administration of the anti-BlyS antibody has ceased. In a further embodiment, the anti-BlyS antibody is systematically administered for a maximum period of 6 months after the last dose of the anti-CD20 antibody, for example, it is administered for no longer than 3 months or no longer than 4 months after the last dose of the anti-CD20 antibody.

The dosing of the anti-CD20 antibody after the anti-BLyS antibody allows the opportunity for the B cells to mobilize from lymphoid tissues. The mobilization of B cells is known to occur 1 week after anti-BLyS antibody dosing however, in order to allow suitable time for the anti-BLyS antibody to take effect whilst not simultaneously administering an anti-CD20 antibody to patients being given background immunosuppressants, a carefully balanced dosage regimen is required.

For example, in one embodiment the immunosuppressants will be discontinued at week 4 prior to the first dose of anti-CD20 antibody (e.g. rituximab) after 4 weeks of anti-BlyS antibody (e.g. belimumab) treatment.

According to another aspect of the invention, there is provided the use of a BlyS antagonist in the manufacture of a medicament for the treatment of Long Covid and/or post-acute sequelae SARS-COV-2 infection (PASC) or alternatively for use in the treatment of an autoimmune condition induced following a virus infection. In a yet further aspect, there is provided a pharmaceutical composition for use in the treatment of Long Covid and/or post-acute sequelae SARS-COV-2 infection (PASC) or alternatively for use in the treatment of an autoimmune condition induced following a viral infection, said pharmaceutical composition comprising a BlyS antagonist. As will be readily appreciated, such medicaments and/or pharmaceutical compositions may be used, such as administered, according to and in any methods or embodiments described herein.

According to such aspects, the medicament and/or pharmaceutical composition comprise the BlyS antagonist, such as an anti-BlyS antibody, as defined herein and one or more pharmaceutically acceptable excipients or carriers. In some embodiments, the medicament and/or pharmaceutical composition comprises a therapeutically effective amount of BlyS antagonist. Typically, such pharmaceutical compositions comprise a pharmaceutically acceptable carrier as known and called for by acceptable pharmaceutical practice. Examples of such carriers include sterilized carriers, such as saline, Ringers solution, or dextrose solution, optionally buffered with suitable buffers to a pH within a range of 5 to 8. Pharmaceutical compositions and medicaments may be administered by injection or infusion as described herein (e.g. intravenous, intraperitoneal, intradermal, subcutaneous, intramuscular or intraportal). Such compositions and medicaments are suitably free of visible particulate matter. Pharmaceutical compositions and medicaments may comprise an amount of BlyS antagonist, such as an anti-BlyS antibody as defined and specified herein.

Methods for the preparation of such pharmaceutical compositions and medicaments are well known to those skilled in the art. Pharmaceutical compositions and medicaments may comprise an amount of BlyS antagonist in unit dosage form, optionally together with instructions for use. Pharmaceutical compositions and medicaments may be lyophilized (freeze dried) for reconstitution prior to administration according to methods well known or apparent to those skilled in the art. Where antibodies have an IgG1 isotype, a chelator of copper, such as citrate (e.g. sodium citrate) or EDTA or histidine, may be added to the pharmaceutical composition or medicament to reduce the degree of copper-mediated degradation of antibodies of this isotype. Pharmaceutical compositions and medicaments may also comprise a solubilizer, such as arginine, a surfactant/anti-aggregation agent such as polysorbate 80, and an inert gas such as nitrogen to replace vial headspace oxygen.

### Covid-19

At the time of writing SARS-COV-2 is a beta coronavirus having greater than 95% sequence identity at the RNA level with any one of the sequences deposited in the China National Microbiological Data Centre under accession number NMDC10013002. In other embodiments, it has greater than 96% sequence identity, greater than 97% sequence identity, greater than 98% sequence identity or greater than 99% sequence identity at the RNA level with any one of the sequences deposited in the China National Microbiological Data Centre under accession number NMDC10013002. The definition is intended to cover all strains of SARS-COV-2 including the L and S strains (S strain has a T at position 8782 and a C at position 28144; L strain has a C at position 8782 and a T at position 28144, with numbering relating to the reference genome of SARS-COV-2 (NC_045512)) and including the O, V, G, HG, GR and GV strains. It is appreciated that the virus may change over time and new classifications are contemplated.

COVID-19 refers to the collection of symptoms exhibited by patients infected with any strain of SARS-COV-2. Symptoms typically include cough, fever and shortness of breath (dyspnoea). Approximately 10-15% of patients diagnosed with COVID-19 experience severe disease involving respiratory problems that can require hospitalisation and intensive care, with an additional 5% of patients becoming critically ill. Age is widely recognised as a significant risk factor for severe COVID-19 disease. Greater disease severity and increased mortality are consistently observed in older patients with severe pulmonary COVID-19. According to the United States' Centers for Disease Control and Prevention, the risk of hospitalization is 5-times greater for patients aged 70 to 74, rising to 8-times higher for patients aged 75 and older. These patients often need breathing interventions including significant oxygen support or mechanical ventilation. The severe respiratory symptoms of COVID-19 are caused by the body's immune system going into overdrive to eliminate the virus and can lead to life-threatening complications or even death.

The reason why older patients are more susceptible to COVID-19 is believed to be partly due to the remodelling of the immune system that occurs during aging, termed “immunesenescence” or “inflammaging”, where an aging immune system may predispose older patients to worse outcomes. Changes in COVID-19 monocyte function and phenotype combined with changes in the adaptive immune system have been proposed to resemble those that occur with normal aging and were hypothesised to be a potential mechanism predisposing older patients to severe COVID-19 disease. Older patients see a decrease in Type 1 IFN and natural killer cell cytokine production, chronic low grade systemic inflammation, frequency of pro-inflammatory monocytes and accumulation of functionally exhausted and senescent CD4+ and CD8+ T cells. Prior to infection, older patients have low grade systemic inflammation (“inflammaging”), so are at a greater risk of developing a pronounced inflammatory response after infection. Furthermore, in older patients the adaptive immune system is more likely to fail or take too long to ramp up, so there is a heavy reliance on the innate immune system which is less successful in clearing viral infections.

Most studies to date have focused on symptoms duration and clinical outcomes in adults hospitalized with severe COVID-19. There are indications in literature that even among symptomatic adults tested in outpatient settings, it might take weeks for resolution of symptoms and return to usual health. Not returning to usual health within 2-3 weeks of testing was reported by approximately one third of COVID-19 patients. Even among young adults aged 18-34 years with no chronic medical conditions, nearly one in five reported that they had not returned to their usual state of health within 3 weeks of infection. In contrast, over 90% of outpatients with influenza recover within approximately 2 weeks of having a positive test. Whilst higher risk factors for acute covid is seen in males and the elderly and those with underlying health conditions Long Covid appears more prevalent in females and younger age groups wherein underlying pre-existing health conditions are not always present. Additionally the severity of the initial infection is often not determinative of Long Covid and/or PASC diagnosis.

In one embodiment, the invention provides a BlyS antagonist for use in treatment wherein a subject is or was previously identified as being infected with SARS-COV-2 by detection of viral RNA from SARS-COV-2 from a specimen obtained from the subject. For example, wherein the subject was identified as being infected with SARS-COV-2 about 4 weeks or about 12 weeks or more than 12 weeks prior to treatment. In one embodiment, the subject is infected or was previously infected with the L strain of SARS-COV-2. In another embodiment, the subject is infected or was previously infected with the S strain of SARS-COV-2. In another embodiment, the subject is infected or was previously infected with the O strain of SARS-COV-2. In another embodiment, the subject is infected or was previously infected with the V strain of SARS-COV-2. In another embodiment, the subject is infected or was previously infected with the G strain of SARS-COV-2. In another embodiment, the subject is infected or was previously infected with the GH strain of SARS-COV-2. In another embodiment, the subject is infected or was previously infected with the GR strain of SARS-COV-2. In another embodiment, the subject is infected or was previously infected with the GV strain of SARS-COV-2. In another embodiment, the subject is infected or was previously infected with the G/452R.V3 strain of SARS-COV-2. In another embodiment, the subject is infected or was previously infected with the British variant of SARS-COV-2 for example the alpha variant and in particular B1.1.7. In another embodiment, the subject is infected or was previously infected with the South African variant of SARS-COV-2. In a further embodiment, the subject is infected or was previously infected with the Brazilian variant of SARS-COV-2. In a yet further embodiment, the subject is infected or was previously infected with the Indian variant of SARS-COV-2. It will be appreciated that as other variants emerge these too are within the scope of the invention.

COVID-19 infection can be classified into 3 grades of increasing severity (Siddiqi & Mehra J Heart Lung Transplant (2020); https://doi.org/10.1016/j.healun.2020.03.012) which differ in their clinical symptoms, clinical signs and potential therapies.

Stage 1: This is the early infection stage and for most people, is associated with mild symptoms such as malaise, high temperature and dry cough. A complete blood count may reveal lymphopenia and neutrophilia. During this stage the virus multiplies and establishes itself within the host, primarily within the respiratory system. Patients who remain within this stage have an excellent prognosis.

Sub-sets of COVID-19 patients commonly exhibit an expansion of pathological extrafollicular B cell populations (IgD-/CD27-double negative, DN) that have previously been described in systemic lupus erythematosus (SLE) patients. Extrafollicular B cells have been associated with autoantibody production in SLE patients and more recently also in a subset of COVID-19 patients. Thus, a break in self tolerance (as has been described in SLE) during SARS-COV-2 infection has become evident by the expansion of extrafollicular B cells and autoantibodies to a variety of autoantigens.

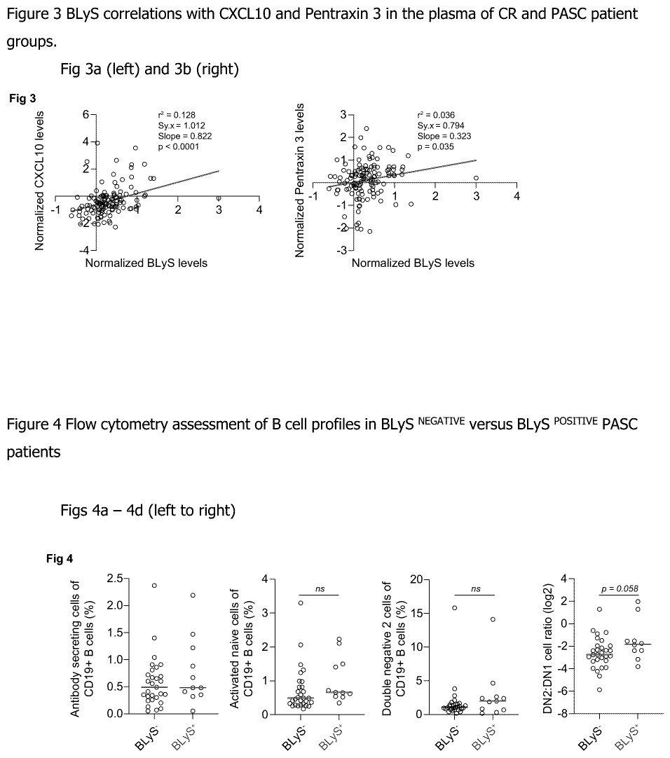

Elevated BLyS levels have been observed during SARS-COV-2 infection (Data provided by the MGH Emergency Department COVID-19 Cohort (Filbin, Goldberg, Hacohen) with Olink Proteomics) although levels of Blys/BAFF are seen to drop back to those of healthy individuals once the acute infection has passed FIG. 3 https://pubmed.nchi.nlm.nih.gov/32668194/. It has been shown in animal models that overexpression of BLyS can promote SLE-like autoimmunity whilst in humans elevated BLyS levels are associated with SLE disease severity; BLyS neutralization can be effective in the treatment of autoantibody-positive SLE.

Since BLyS-driven breaks of self-tolerance have been described in hyper-inflammatory diseases such as SLE, shared pathogenic mechanisms between hyper-inflammatory diseases and COVID-19 may suggest that SARS-COV-2 could act as a triggering factor for the development of autoimmune and/or autoinflammatory dysregulation. Furthermore, patients with pre-existing autoimmune conditions may flare during or following the SARS-COV-2 infection.

Stage 2: This is the pulmonary phase where viral multiplication and localized inflammation in the lung are high. Patients develop viral pneumonia with (Phase 2B) and without (Phase 2A) hypoxia (defined as PaO₂/FiO₂<300 mmHg), abnormal chest imaging, transaminitis and low/normal procalcitonin. Further symptoms include fever and cough. In this stage patients would normally need to be hospitalized for treatment. As COVID-19 progresses, it is important to suppress hyperinflammation to prevent further lung and end-organ damage.

Stage 3: This is the hyperinflammation phase which manifests as an extra pulmonary systemic hyperinflammation syndrome. Cytokines and biomarkers are significantly elevated and T cell counts are decreased, this stage is also associated with acute respiratory distress syndrome, systemic inflammatory response syndrome including cytokine release syndrome, shock and cardiac failure. The prognosis for these patients is poor.

Long-COVID and/or PASC: Despite the differing severity experienced by many patients who are infected by Covid 19 the long-term effects are not restricted to those who needed go to hospital, or even who felt seriously unwell when they first caught the virus.

### Long Covid

In one embodiment, the condition is Long Covid or PASC. Throughout the specification it is to be understood that where the term “Long Covid” is used the embodiments and features of the invention apply equally to Post-acute sequelae SARS-COV-2 infection (PASC). Long-COVID is used to describe the effects of COVID-19 that continue for weeks or months beyond the initial acute phase of the illness. Therefore in a further embodiment Long Covid is used according to any clinically acceptable definition.

In addition to the clinical case definitions, “Long Covid” is commonly used to describe signs and symptoms that continue or develop after acute COVID-19. It includes both ongoing symptomatic COVID-19 and post-COVID-19 syndrome (defined below).

The term “acute COVID-19” as used herein refer to the signs and symptoms of COVID-19 which may last for up to 4 weeks.

The term “ongoing symptomatic COVID-19” refers to signs and symptoms of COVID-19 which present from 4 to 12 weeks.

The National Institute for Health and Care Excellence (NICE) in the UK and the World Health Organisation defines Long Covid as lasting for more than 12 weeks, although “Long Covid” is widely attributed to symptoms that last for more than eight weeks. The CDC defines Long Covid as symptoms lasting for more than 4 weeks after infection. The diagnosis is often made because the symptoms are not explained by an alternative diagnosis. It usually presents with clusters of symptoms, often overlapping, which can fluctuate and change over time and can affect any system in the body.

One group of common symptoms are mainly respiratory, such as a cough and feeling breathless (dyspnea) but also include fatigue and headaches. A second group of symptoms affects many parts of the body, including the heart, brain and the gut. For example, heart symptoms such as palpitations or increased heartbeat, as well as pins and needles, numbness and ‘brain fog’ have been commonly reported. Thus, as will be appreciated, many of the symptoms of Long Covid are shared with CFS/ME as described above. In one embodiment there is provided the use of a Blys antagonist for use in the treatment of Long Covid and/or PASC patients wherein the patients are diagnosed as suffering from dyspnea and cough. There are a number of cough-related quality of life scores which have been used in patients with chronic cough due to a variety of conditions. The most commonly used include: Leicester Cough Questionnaire (LCQ), Cough-specific Quality of Life Questionnaire (CQLQ) and the Simple visual analogue scale. Cough can also be measured objectively with a cough counter device or app which records the number of coughs per 24 hour period such as the Leicester Cough Monitor and the VitaloJAK. Therefore in one embodiment a patient is diagnosed as suffering from a cough according to any known method in the art such as those described in jtd-12-09-5207.pdf (nih.gov) A Review Article on the 3rd International Cough Conference “The present and future of cough counting tools” Hall et al.2020.

Although Dyspnea is a subjective symptom functional exercise tests, such as the 6 minute walk test, can give some objective data. A number of dyspnea scores can be used, with some of the most well-established being: MRC Breathlessness Scale—a simple scale from 1-5 assessing functional disability due to breathlessness Baseline Dyspnea Index—scored from 0-12 can also be used with the Transition Dyspnea Index for follow-up assessments and the Borg Dyspnea Scale—used to quantify breathlessness during exercise from 0-10 (e.g. as part of an exercise test). In one embodiment the patient is diagnosed as suffering from dyspnea when measured according to any known method in the art.

The St George's Respiratory Questionnaire is a well-established quality of life score that assesses the impact of respiratory disease on overall health/quality of life. It includes questions on both cough and breathlessness. The St. George's Respiratory Questionnaire is a self-completed test with three components—symptoms (distress caused by respiratory symptoms), activity (disturbance in daily activities), and impact (psychosocial function)—summed to give a total score of overall health status. This is the most validated health status tool in COPD. The mean score in healthy adults is approx. 8 to 12 (Ferrer E R J 2002, Weatherall E R J 2009). A change of 4 points is generally considered the minimal clinically important difference. Several studies (e.g. Jones 2011, Wacker 2016, Kharbanda 2021) have looked at average SGRQ scores in relation to COPD severity, as measured by the GOLD stage:

| GOLD | |
|---|---|
| COPD Stage | Mean SGRQ Sore |
| 1 (mild) | 28.0-34.9 |
| 2 (moderate) | 38.7-41.9 |
| 3 (severe) | 48.6-55.1 |
| 4 (very severe) | 58.4-69.6 |

The paper from Wacker et al has some detailed comparisons of outcome measures in relation to COPD severity including SGRQ, EQ5D etc: Wacker B M C Pulm Med 2016

In one embodiment the patients are diagnosed as suffering from dyspnea and cough according to the St George's Respiratory Questionnaire measures. In a further embodiment the patient is diagnosed as having an SGRQ score of at least 25 for example 28 to 34.9, or at least 35 for example 38.7 to 41.9 or for example at least 45 for example 48.6 to 55.1 or for example at least 55 for example 58.4 to 69.6 or for example at least 60.

Recent development and early validation of 2 novel Patient reported outcomes for Long COVID and/or PASC include the Symptom Burden Questionnaire (SBQ-LC), 17 independent scales, with promising psychometric properties, has undergone Rasch analysis (Hughes, Sarah E., et al. “Development and validation of the Symptom Burden Questionnaire™ for Long COVID: a Rasch analysis.” medRxiv (2022)) and COVID-19 Yorkshire Rehabilitation Scale (C19-YRS) currently deployed in UK community rehabilitation setting and 26 NHS Long COVID centers (O'Connor, Rory J., et al. “The COVID-19 Yorkshire Rehabilitation Scale (C19-YRS): application and psychometric analysis in a post-COVID-19 syndrome cohort.” Journal of Medical Virology 94.3 (2022): 1027-1034).

As yet there is no clearly identified single common mechanistic pathway that impacts all Long Covid or post-acute sequelae SARS-COV-2 infection (PASC) symptoms. Long Covid or post-acute sequelae SARS-COV-2 infection (PASC) is a variably defined syndrome with heterogeneous symptomatology; many focus on symptoms persisting >12 weeks. although the Center for Disease Control and Prevention, (CDC) in 2021 defined it as a range of new, returning, or ongoing health problems people can experience four or more weeks following initial SARS-COV-2 infection. At least 5 putative mechanisms including SARS-COV-2 viral persistence, autoantibody formation, deficits in resolution of inflammation, mitochondrial dysfunction, latent viral (Epstein Bar Virus (EBV), other) reactivation have now been identified.

As described herein before, severe/critical COVID-19 patients have been identified which display clinical autoreactivities such as anti-nuclear antibodies (ANAs), autoantibodies against phospholipids, type-I interferons, rheumatoid factor (RF), and other self antigens. Furthermore, circulating B cells in critically ill patients with COVID-19 are phenotypically similar to the extrafollicular B cells that were previously identified in patients with autoimmune diseases such as SLE. Therefore, in one embodiment the Long Covid and/or PASC patients are characterised by the presence of autoantibodies. These include antibodies to cytokines, interferons, chemokines and leukocytes, which could directly affect the course of antiviral immunity by antagonizing innate antiviral responses, thus impacting disease progression as well as antibodies to tissue-specific antigens expressed in the central nervous system, vasculature, connective tissues, cardiac tissue, hepatic tissue and intestinal tract, which could potentially cause antibody-mediated organ damage. Thus, in certain embodiments, the autoimmune condition is characterised by the presence of autoantibodies with the potential for driving de novo immune-mediated inflammatory diseases. In a further embodiment, the titre and/or levels of anti-nuclear antibodies (ANAs) and/or anti-rheumatoid factor (RF) antibodies are those seen in systemic lupus erythematosus (SLE). Such autoantibodies may be present in any organ or tissue of the subject but due to the potentially high levels associated with the autoimmune condition will be found in the blood of said subject. Thus, in a further embodiment the autoantibodies are present in the blood of the subject.

In a yet further embodiment, the autoantibodies are selected from: anti-nuclear antibodies (ANAs), anti-rheumatoid factor (RF) antibodies, anti-double-standed DNA (dsDNA) antibodies, anti-extra nuclear antigens (ENA) antibodies, anti-ribosomal-P antibodies, anti-RNP-70 antibodies, anti-Sjögren's-syndrome-related antigen A (SS-A) and/or anti-Sjögren's-syndrome-related antigen B (SS-B) antibodies, anti-Sm antibodies, anti-phospholipid antibodies, anti-anterior cruciate ligament (ACL) antibodies, anti-lupus anticoagulant (LAC) antibodies, and/or anti-beta-2-glycoprotein-1 antibodies. In one embodiment, the titre of anti-nuclear antibodies (ANAs) is greater than 1:80 and/or anti-rheumatoid factor (RF) antibodies is greater than 20 IU/mL. In a further embodiment, the titre of ANAs is greater than 1:80 and the titre of RF antibodies is greater than 20 IU/mL. In a further embodiment the patients to be treated test positive for at least 2 or more or 3 or more subsets of autoantibodies in the blood. In a further embodiment the patients test positive for at least anti nuclear antibodies (ANA) and/or anti-phospholipid antibodies.

Patients suffering with Long Covid and/or PASC experience a number of symptoms as mentioned herein before, including breathlessness, chest pain or tightness, chronic fatigue, problems with memory and concentration known as “brain fog”, depression, anxiety and stress. The condition usually presents with clusters of symptoms, often overlapping, which may change over time and can affect any system within the body. It also notes that many people can also experience generalised pain, rashes, heart palpitations, tinnitus and ear problems, dizziness, pins and needles, joint pain, feeling sick, diarrhoea, stomach aches, loss of appetite, changes to sense of smell or taste, persisting high temperature and psychiatric problems. Measures, such as the SRI, health-related quality of life (SF-36, which measures changes in the quality of life in several physical and mental health domains) and the Functional Assessment of Chronic Illness Therapy (FACIT)-Fatigue (which measures fatigue) may be used to assess a patient with Long Covid. A mean change from baseline in the physical and mental components of the SF-36 may be considered an improvement, including measurements of bodily pain, general health, physical functioning, role physical, social functioning, and vitality. Thus, in one embodiment, treatment as described herein, such as treatment for Long Covid, includes an improvement SF-35 measure. In a further embodiment, treatment, such as treatment for Long Covid, includes an improvement in the FACIT-F score, showing that the treatment eased symptoms of fatigue.

### Autoimmune Conditions

In one aspect of the present invention, the BlyS antagonist as described herein, such as an anti-BlyS antibody, is provided for use in the treatment of an autoimmune condition induced following a viral infection. Throughout the specification the terminology “induced following a viral infection” refers to the viral infection as the trigger or suspected causative agent in the absence of any other, for a worsening medical state. For example the viral infection is responsible for the autoreactivity or break in tolerance that either causes a previously healthy individual to develop an autoimmune condition or causes a previously diagnosed individual with an autoimmune condition to worsen or flare.

In one embodiment the autoimmune condition is de novo.

In another embodiment the autoimmune condition is a pre-existing condition, such as an autoimmune disease or disorder with which the subject has previously been diagnosed or which has previously been identified in the subject prior to viral infection. According to this embodiment, the pre-existing condition is worsened upon viral infection or is re-activated if the subject is considered to be in remission from the pre-existing autoimmune condition. Such re-activation is also known as “flare” or a “flare up”. In one embodiment, the subject has a pre-existing autoimmune condition. In a further embodiment, the pre-existing autoimmune condition is present in the subject prior to viral infection. In a yet further embodiment, the pre-existing autoimmune condition is modified upon viral infection, for example changed such that additional/alternative autoantibodies may be detected in the subject.

In another aspect, there is provided a method for the treatment of an autoimmune condition induced following a viral infection in a subject in need thereof, said method comprising administering to said subject a therapeutically effective amount of a BlyS antagonist, such as an anti-BlyS antibody.

In one aspect, there is provided a method of treating Long Covid and/or PASC in a subject comprising the steps of:
i) optionally obtaining a sample from the subject;
ii) testing the sample for the levels of serum cytokines, Blys, IFN-β, PTX3, IFN-lambda 2/3 and/or IL-6;
iii) optionally comparing/determining levels of any of the results of step ii) with a healthy reference level;
iv) if the levels are at least 2 fold higher than the reference level, then administering to the subject a therapeutically effective amount of a Blys antagonist.

In another aspect, there is provided a method of treating Long Covid and/or PASC in a subject comprising the steps of:
i) comparing the levels of serum cytokines, Blys, IFN-β, PTX3, IFN-lambda 2/3 and/or IL-6 of the subject with a healthy reference level;
ii) if the levels are at least 2 fold higher than the reference level, then administering to the subject a therapeutically effective amount of a Blys antagonist.

In another aspect, there is provided a method of treating Long Covid and/or PASC in a subject comprising administering to the subject a therapeutically effective amount of a Blys antagonist, wherein the levels of serum cytokines, Blys, IFN-β, PTX3, IFN-lambda 2/3 and/or IL-6 of the subject are at least 2 fold higher than a healthy reference level.

A healthy reference level as used throughout is used to define levels expected by a physician to indicate a healthy patient based on well known methods in the art.

As described herein before and in the review by Smatti et al. (Smatti, M. K. et al., Viruses (2019); https://doi.org/10.3390/v11080762) viruses and viral infections have long been shown to be involved in and modify autoimmune diseases and conditions. This can occur even in distant and seemingly unrelated organs and tissues, such as for example the onset of type 1 diabetes following influenza A virus infection.

In one embodiment, the autoimmune condition is chronic. In another embodiment, the subject is suspected or identified with, such as diagnosed with, a chronic autoimmune condition. Such chronic conditions will be understood to affect the subject for a period of time, such as an extended period of time, after the acute phase or symptoms of a disease have passed. For example, following an initial virus infection and acute phase of said infection, the subject may continue to feel the effects of said infection or develop new symptoms, such as fatigue and/or nausea. In a particular embodiment, the autoimmune condition persists or presents 4 weeks following infection or 8 week or 12 weeks following initial infection. Thus, in one embodiment, the autoimmune condition presents at or after 4 weeks following a viral infection or 8 or 12 weeks following a viral infection, such as SARS-COV-2 infection.

In some embodiments, the autoimmune condition is chronic fatigue syndrome (CFS) or myalgic encephalomyelitis (ME). CFS/ME is a complex disorder of unknown cause which is characterised by extreme fatigue lasting for 6 months or more, wherein the symptoms of fatigue can be worsened by exercise but do not improve with rest. Although the causes of CFS/ME are unknown and there is currently no treatment for this condition, several symptoms are shared with those of autoimmune conditions such as systemic lupus erythematosus (SLE) and include the following: extreme exhaustion; non-restorative sleep; brain fog/cognitive impairment; joint pain; inflamed lymph nodes; persistent sore throat; severe headache; neurological abnormalities; complete organ system shutdown; and sensitivity to light, sound, odors, chemicals, foods, and medications.

Thus, in other embodiments the autoimmune condition is a de novo immune-mediated inflammatory disease, such as:
Systemic lupus erythematosus (SLE): wherein patients meet at least 4 of 11 modified American College of Rheumatology (ACR) (1997) Revised Criteria for the Classification of Systemic Lupus Erythematosus or Systemic Lupus International Collaborating Clinics (SLICC) classification criteria for systemic lupus erythematosus);
Anti-neutrophil cytoplasmic antibody associated vasculitis (AAV): wherein patients meet the Revised 1990 ACR criteria for Granulomatosis with polyangiitis (GPA) or the 2012 Chapel Hill Nomenclature for microscopic polyangiitis (MPA);
Idiopathic inflammatory myopathies (IIM): wherein patients meet the 2017 EULAR/ACR Classification Criteria for Adult and Juvenile idiopathic inflammatory myopathies and their major subgroups;
Primary Sjogren's syndrome (SS): wherein patients meet the 2016 ACR criteria;
Progressive systemic sclerosis (PSS): wherein the ACR/EULAR 2013 Classification criteria are met;
Immunologically mediated kidney diseases (IKD): wherein presence of the disease is based on results from kidney biopsy consistent with immunologically mediated nephrotic syndrome and/or glomerulonephritis;
Multisystem inflammatory disease of children (MIS-C) or the multisystem inflammatory disease of adults (MIS-A).
Rheumatoid arthritis (RA): wherein patients meet the 2010 ACR/EULAR Classification Criteria; and/or
Systemic autoimmunity syndrome not otherwise specified (SA-NOS): wherein patients hare characterised as having no autoimmune disease but with evidence of autoantibodies and clinical features suggestive of a systemic autoimmune disorder.

Thus, in some embodiments, the autoimmune condition is induced or worsened following a viral infection. In further embodiments, the viral infection is an enteric virus infection, a herpes virus infection, an influenza infection or a coronavirus infection. As described hereinbefore and in Smatti et al., such viral infections may lead to or contribute to the development of autoimmune conditions, although the specific mechanisms behind this are unknown. Therefore, the viral infection may be by any virus. In one embodiment the virus is Epstein Barr Virus or reactivation of such a virus. In certain embodiments, the enteric virus infection is a Coxsackie B virus (CVB) or rotavirus infection, wherein the influenza virus infection is an influenza A infection, or wherein the coronavirus infection is a SARS-COV-2 infection. In a particular embodiment, the viral infection is a SARS-COV-2 infection, or a COVID-19 infection. In one embodiment the trigger is the reactivation of the latent virus and not simply the initial infection.

In one embodiment, the autoimmune condition is characterised by a serum ferritin level above 150 ng/ml in females and above 300 ng/ml in males in a subject. In a further embodiment, the autoimmune condition is characterised by a C-reactive protein level above 10 mg/L in a subject. In a yet further embodiment, the autoimmune condition is characterised by a serum ferritin level above 150 ng/ml in females or above 300 ng/ml in males in a subject, and a C-reactive protein level above 10 mg/L in the subject. In another embodiment, the autoimmune condition is characterised by the subject previously having a serum ferritin level above 150 ng/ml in females or above 300 ng/ml in males and/or a C-reactive protein level above 10 mg/L in the subject during the viral infection.

### Treatment of COVID-19

Treatment of COVID-19 (for example severe pulmonary COVID-19), cytokine release syndrome, acute respiratory distress syndrome (ARDS), a cytokine storm or myeloid cell driven vasculitis refers to a reduction in the severity or duration of the symptoms of the disease, for example using the WHO committee ordinal scale (given in Table B below) that measures illness severity over time. All these are considered to be acute manifestation of the disease.

TABLE B

| | |
|---|---|
| 1. | Non-hospitalized, no limitation of activity |
| 2. | Non-hospitalized, limitation of activity¹ |
| 3. | Hospitalized, no oxygen therapy |
| 4. | Hospitalized, low-flow oxygen by mask or nasal prongs |
| [symbol] | Hospitalized, high-flow oxygen (≥15 L/min), CPAP², BIPAP³, non-invasive ventilation |
| 6. | Hospitalized, intubation and mechanical ventilation (no additional organ support) |
| [symbol] | Hospitalized, mechanical ventilation plus additional organ support (e.g. vasopressors, RRT⁴, |
| | EC MO⁵) |
| 8. | Death |

¹report as category 4 if using oxygen at home,
²continuous positive airway pressure,
³bilevel positive airway pressure,
⁴renal replacement therapy,
⁵extracorporeal membrane oxygenation. [symbol]
indicates data missing or illegible when filed

The WHO ordinal scale has been widely used in COVID-19 studies and has also been used in the past (2019) in studies of critically-ill patients with influenza virus infection.

In one embodiment treatment of the acute phase of disease is a reduction in illness severity based on the WHO committee ordinal scale. In other embodiments, treatment includes amelioration. In another embodiment, treatment results in an improvement in symptoms. In one embodiment symptoms of the disease include levels of biological markers of systemic inflammation above normal levels and oxygenation impairment. In another embodiment improvement results in the subject transitioning to low flow oxygen (<15 L/min) by mask or nasal prongs or no oxygen therapy.

Alternatively, treatment of the acute phase of the disease could be a reduction in viral load. Viral load may be measured by a suitable quantitative RT-PCR assay from a specimen from the patient. In one embodiment, the specimen may be a specimen from the upper or lower respiratory tract (such as a nasopharyngeal or oropharyngeal swab, sputum, lower respiratory tract aspirates, bronchoalveolar lavage, bronchial biopsy, transbronchial biopsy and nasopharyngeal wash/spirate or nasal aspirate) saliva or plasma. In a more particular embodiment, the specimen is saliva. The protocols of a number of quantitative RT-PCR assays are published on https://www.who.int/emergencies/diseases/novel-coronavirus-2019/technical-guidance/laboratory-guidance. In addition, Corman and colleagues have published primers and probes for use in such assays (Corman et al., European communicable disease bulletin (2020), https://doi.org/10.2807/1560-791Z). In one embodiment, the COVID-19 RdRp/Hel assay is used. This has been validated with clinical specimens and has a limit of detection of 1.8 TCID₅₀/ml with genomic RNA and 11.2 RNA copies/reaction with in vitro RNA transcripts (Chan et al., J Clin Microbiol. (2020), https://doi.org/10.1128/JCM.00310-20). Viral titre may be measured by assays well known in the art.

In another embodiment, treatment of an autoimmune condition induced or worsened following a viral infection and/or Long Covid includes amelioration. In another embodiment, treatment results in an improvement in symptoms. Ameloration and/or improvement of symptoms include reduction in fatigue, improved sleep, reduced brain fog/cognitive impairment (e.g. reduced headache), reduced joint pain, a reduction in the inflammation of lymph nodes, fewer/reduced neurological abnormalities, prevention of complete organ system shutdown, and/or reduced sensitivity to external sources, such as light, foods or medications. In a further embodiment, treatment includes a reduction in the levels of biological markers of systemic inflammation, such as in the blood, for example to normal levels. In a yet further embodiment, treatment includes the reduction in titre and/or levels of autoantibodies seen in SLE, such as the titre and/or levels of anti-nuclear antibodies (ANAs) and/or anti-rheumatoid factor (RF) antibodies. In a still further embodiment, treatment includes the reduction in the titre of anti-nuclear antibodies (ANAs) to below 1:80 and/or anti-rheumatoid factor (RF) antibodies to less than 20 IU/mL. In a further embodiment, the titre of ANAs is reduced to less than 1:80 and the titre of RF antibodies is reduced to less than 20 IU/mL following treatment.

The term “amelioration” as used herein is the prevention or reduction in the severity or duration of the symptoms of the disease, for example using the WHO committee ordinal scale (given in Table 2 above) in the case of acute disease that measures illness severity over time. Amelioration includes, but does not require, complete recovery or complete prevention of a disease or symptoms thereof.

The term “prevention” as used herein refers to complete prevention of symptoms of the disease, for example cytokine release syndrome, acute respiratory distress syndrome, myeloid cell driven vasculitis, an increase in biological markers of systemic inflammation above normal levels or oxygenation impairment.

The term “cytokine release syndrome” (CRS) as used herein is a form of systemic inflammatory response syndrome (SIRS) that can be triggered by a variety of factors such as infections and certain drugs. It occurs when the immune system causes an uncontrolled and excessive release of pro-inflammatory cytokines. This sudden release in such large quantities can cause multisystem organ failure and death. “Cytokine release syndrome” also includes the situation where symptoms are due to treatment and are delayed until days or weeks after said treatment. In one embodiment, cytokine release syndrome is a pronounced inflammatory response.

Drugs that may trigger cytokine release syndrome include immunotherapeutics such as monoclonal antibodies, bispecific antibodies, antibody drug conjugates, immune checkpoint inhibitors, T cell engaging single chain antibody constructs, chimeric antigen receptor (CAR) T cells and T cell receptor (TCR) T cells. In one embodiment the cytokine release syndrome is the consequence of immunotherapy.

CRS clinically manifests when large numbers of lymphocytes (B cells, T cells, and/or natural killer cells) and/or myeloid cells (macrophages, dendritic cells, and monocytes) become activated and release inflammatory cytokines. Key cytokines include TNFα, IFNγ, IL-1β, IL-2, IL-6, IL-8 and IL-10. IL-6 in particular, is emerging as a central mediator of toxicity in CRS. Symptoms of CRS include fever, nausea, fatigue, headache, myalgias, malaise, rigors, hypotension, unexpected oxygen requirement and/or organ toxicity. Respiratory symptoms include rapid breathing and cough in the milder stages, but can progress to ARDS with laboured breathing and hypoxemia. In one embodiment ARDS is the consequence of cytokine release syndrome. In another embodiment ARDS is the consequence of immunotherapy.

The term “pronounced inflammatory response” as used herein refers to a systemic inflammatory response or cytokine release syndrome wherein the patients' levels of cytokines are elevated, but not to the levels observed in ARDS. In one embodiment the levels of IL-6 in the patient's plasma are from 6 to 170 pg/mL.

The term “myeloid cell driven vasculitis” as used herein refers to a group of conditions characterized by blood vessel inflammation wherein the inflammation is caused and propagated by cells of myeloid lineage, for example monocytes and neutrophils. In one embodiment the myeloid driven vasculitis is predominantly in the lungs.

The term “cytokine storm” as used herein is a form of systemic inflammatory response syndrome (SIRS) that can be triggered by a variety of factors such as infections and certain drugs. It occurs when the immune system causes an uncontrolled and excessive release of pro-inflammatory cytokines. This sudden release in such large quantities can cause multisystem organ failure and death.

The term “COVID-19 pneumonia” as used herein is defined as COVID-19 wherein the patient is hospitalized due to diagnosis of pneumonia (chest X-ray or CT scan consistent with COVID-19 infection).

The term “severe pulmonary COVID-19” as used herein is defined as COVID-19 pneumonia where the patient has developed oxygenation impairment. In one embodiment the term “severe pulmonary COVID-19” as used herein is defined as COVID-19 wherein the patient is hospitalized due to diagnosis of pneumonia (chest X-ray or CT scan consistent with COVID-19 infection), has developed oxygenation impairment and has an increase in C-reactive protein (CRP) and/or serum ferritin above upper limit of normal.

In another embodiment, the term “severe pulmonary COVID-19” as used herein is defined as COVID-19 wherein the patient is hospitalized due to diagnosis of pneumonia (chest X-ray or CT scan consistent with COVID-19 infection) and has developed oxygenation impairment defined as:
peripheral capillary oxygen saturation (SpO₂) less than 93% on room air and high flow oxygen, or continuous positive airway pressure (CPAP)/Bilevel Positive Airway Pressure (BiPAP), or non-invasive ventilation (NIV), or mechanical ventilation; and have
increased C-reactive protein (CRP) above upper limit of normal and/or serum ferritin above upper limit of normal.

In another embodiment, the term “severe pulmonary COVID-19” as used herein is defined as COVID-19 wherein the patient is hospitalized due to diagnosis of pneumonia (chest X-ray or CT scan consistent with COVID-19 infection) and has developed oxygenation impairment defined as: peripheral capillary oxygen saturation (SpO₂)≤93% on room air, the patient is on high-flow oxygen (≥15 L/min) and/or on non-invasive ventilation (NIV, continuous positive airway pressure (CPAP)/Bilevel Positive Airway Pressure (BiPAP)) or mechanical ventilation; and has increased C-reactive protein (CRP) above upper limit of normal and/or serum ferritin above upper limit of normal.

The term “oxygenation impairment” as used herein is defined as peripheral capillary oxygen saturation (SpO₂)≤93% on room air. In another embodiment, “oxygenation impairment” as used herein is defined as peripheral capillary oxygen saturation (SpO₂) less than 93% on room air and high flow oxygen. In another embodiment, “oxygenation impairment” as used herein is defined as requiring an intervention such as: continuous positive airway pressure (CPAP); bilevel positive airway pressure (BiPAP); non-invasive ventilation (NIV); and/or intubation and mechanical ventilation. In another embodiment “oxygenation impairment” as used herein is defined as peripheral capillary oxygen saturation (SpO₂)≤93% on room air, on high-flow oxygen (≥15 L/min) and/or on non-invasive ventilation (NIV), continuous positive airway pressure (CPAP), Bilevel Positive Airway Pressure (BiPAP) or mechanical ventilation.

The term “high flow oxygen” as used herein is defined as ≥15 L/min.

C-reactive protein (CRP) and/or serum ferritin are biological markers of systemic inflammation. In one embodiment, the biological marker of systemic inflammation is C-reactive protein (CRP). In another embodiment, the biological marker of systemic inflammation is serum ferritin.

The term “above upper limit of normal” as used herein is defined as an amount greater than the level of a healthy individual of a similar age. For example, “above upper limit of normal” for serum ferritin is above 150 ng/ml in females and above 300 ng/ml in males; and “above upper limit of normal” for C-reactive protein is 10 mg/L and above. In one embodiment, the “upper limit of normal” for C-reactive protein is 10 mg/L. In another embodiment the “upper limit of normal” for serum ferritin is 300 ng/mg in a male patient or 150 ng/mg in a female patient.

In one embodiment, a normal level of C-reactive protein is less than 10 mg/L in the blood. In another embodiment, a normal level of serum ferritin before treatment is less than 300 ng/mg in the blood of a male patient or less than 150 ng/mg in the blood of a female patient. In another embodiment, the level of C-reactive protein before treatment is greater than 10 mg/L in the blood. In another embodiment, the level of serum ferritin before treatment is greater than 300 ng/mg in the blood of a male patient or greater than 150 ng/mg in the blood of a female patient. In another embodiment, the level of C-reactive protein after treatment is less than or equal to 10 mg/L in the blood. In another embodiment, the level of serum ferritin after treatment is less than or equal to 300 ng/mg in the blood of a male patient or less than or equal to 150 ng/mg in the blood of a female patient.

The term “acute respiratory distress syndrome” or “ARDS” is well known in the art and is a type of respiratory failure characterized by rapid onset of widespread inflammation in the lungs.

### Myeloid Cell Driven Vasculitis

It has been reported that levels of some inflammatory mediators, including IL-6, are elevated in COVID-19, but are typically ten times lower than those reported in acute respiratory distress syndrome (ARDS) and sepsis, suggesting that other factors may play a major role in COVID-19 severity (Thwaites R. et al., medRxiv (2020); https://doi.org/10.1101/2020.10.08.20209411). Data collected from serial plasma samples taken from 619 patients hospitalised with COVID-19 through the prospective multicentre ISARIC cohort study found elevated levels of D-dimer (a fibrin degradation product, implicating thrombosis), angiopoietin-2 (a marker of endothelial injury), and prothrombotic mediators, thrombomodulin, vWF-A2, and endothelin-1 in hospitalized patients relative to control groups (Thwaites, et al. (2020)).

Arteritis has been identified in the lung of severe COVID-19 patients, and further characterized as a monocyte/myeloid-rich vasculitis that occurred together with an influx of macrophage/monocyte lineage cells into the pulmonary parenchyma. Moreover, post-mortem findings in fatal COVID-19 disease have shown the inflammatory infiltrate in lungs to consist of high levels of macrophages and neutrophils.

Given reports of the association between COVID-19 mortality and pulmonary vasculitis, experts in the field now believe that endothelial injury may be a feature of COVID-19, potentially triggering coagulation and the thrombotic complications common in severe disease. Thrombi in pulmonary vessels has been reported in fatal cases of SARS, ARDS and influenza A virus infection but the frequency in COVID-19 appears to be nearly a log order higher than ARDS and may be due to the distinct endothelial injury pathways.

### Further Therapeutic Uses

In one aspect, the invention provides a BlyS antagonist, such as an anti-BlyS antibody, for use in the treatment or prevention of severe pulmonary COVID-19, cytokine release syndrome (CRS), acute respiratory distress syndrome (ARDS), a cytokine storm and/or myeloid cell driven vasculitis. In another embodiment there is provided a BlyS antagonist for use in the treatment or prevention of ongoing symptomatic COVID-19.

In one aspect, the invention provides a BlyS antagonist for use in the treatment or prevention of severe pulmonary COVID-19, cytokine release syndrome (CRS), acute respiratory distress syndrome (ARDS), a cytokine storm, myeloid cell driven vasculitis, a chronic autoimmune condition and/or Long Covid wherein illness is caused by a coronavirus. In one embodiment, the coronavirus is SARS-COV-2.

In one embodiment, the BlyS antagonist is administered to the subject in need thereof once peripheral capillary oxygen saturation (SpO₂) falls to 95% or less on room air and high flow oxygen. In another embodiment, the subject in need thereof is on low-flow oxygen by mask and nasal prongs. In a further embodiment, the subject in need thereof is on high-flow oxygen, CPAP, BIPAP or non-invasive ventilation. In another embodiment, the subject in need thereof is intubated and on mechanical ventilation. In a yet further embodiment, the subject in need thereof is on mechanical ventilation with additional organ support.

In another embodiment the BlyS antagonist is administered to the subject in need thereof once the subject has an increase in C-reactive protein (CRP) and/or serum ferritin above upper limit of normal. Thus, in one embodiment the BlyS antagonist, such as an anti-BlyS antibody, is administered to a female subject having a serum ferritin level above 150 ng/ml or a male subject having a serum ferritin level above 300 ng/ml. In a further embodiment, the BlyS antagonist is administered to a subject having a C-reactive protein level above 10 mg/L. In a yet further embodiment, the BlyS antagonist is administered to a female subject having a serum ferritin level above 150 ng/ml and a C-reactive protein level above 10 mg/L, or is administered to a male subject having a serum ferritin level above 300 ng/ml and a C-reactive protein level above 10 mg/L. In certain embodiments, the levels described herein are in the blood of the subject.

In other embodiments, treatment of COVID-19 pneumonia or severe pulmonary COVID-19 is initiated within 24 hours of hospitalization due to diagnosis of pneumonia and the onset of oxygenation impairment. In another embodiment, treatment is initiated within 24 hours of the onset of pneumonia. In a further embodiment, treatment is initiated within 24 hours of the onset of oxygenation impairment. In a yet further embodiment, treatment is initiated within 24 hours of the onset of ARDS.

In one embodiment, the subject has COVID-19 pneumonia. In another embodiment the subject has severe pulmonary COVID-19. In a more particular embodiment, the subject has a MuLBSTA score of ≥12, or a CURB-65 score of ≥2 or a PSI score ≥70. In other embodiments, the subject meets one or more of the following criteria: pulse ≥125/minute, respiratory rate >30/minute, blood oxygen saturation≤93%, PaO₂/FiO₂ ratio <300 mmHg, peripheral blood lymphocyte count <0.8·10⁹/L, systolic blood pressure <90 mmHg, temperature <35 or ≥40° C., arterial pH<7.35, blood urea nitrogen ≥30 mg/dl, partial pressure of arterial O₂<60 mmHg, pleural effusion, and/or lung infiltrates >50% of the lung field within 24-48 hours.

In one embodiment, COVID-19 pneumonia is associated with acute respiratory distress disorder. In another embodiment, severe pulmonary COVID-19 is associated with acute respiratory distress disorder. In a more particular embodiment, the subject has a Murray Score of >2. In another embodiment, the subject has a PaO₂/FiO₂ ratio≤200 mmHg. In a more particular embodiment, the subject has a PaO₂/FiO₂ ratio≤100 mmHg. In another embodiment, the subject has a corrected expired volume per minute ≥10 L/min. In another embodiment, the subject has respiratory system compliance≤40 mL/cm H₂O. In another embodiment, the subject has positive end-expiratory pressure ≥10 cm H₂O.

In particular embodiments, the patient is undergoing extra-corporeal membrane oxygen or mechanical ventilation, or non-invasive ventilation, or receiving oxygen supplementation via a nasal cannula or simple mask. Where mechanical ventilation is used, this includes use of low tidal volumes (<6 ml/kg ideal body weight) and airway pressures (plateau pressure <30 cmH₂O). Where oxygen supplementation is via a nasal cannula, this may be delivered as 2 to 6 L/minute. Where oxygen supplementation is by a simple mask, this may be delivered at 5 to 10 L/minute.

### Combinations

In one aspect, the BlyS antagonist, such as an ant-BlyS antibody (e.g. belimumab), for use according to the invention is administered as a monotherapy or in combination with other therapies. Thus, in one embodiment, the treatment further comprises administration of an additional therapeutic agent. In one embodiment, the anti-BlyS antibody is co-administered with standard of care medicaments such as, for example, High Dose Corticosteroids (HDCS), Cyclophosphamide (CYC), Azathioprine (AZA) and/or Mycophenolate Mofetil (MMF).

In particular embodiments, the subject is receiving, has received or will receive anti-viral and/or antibiotic treatment. Thus, in a further embodiment, the additional therapeutic agent is an anti-viral and/or antibiotic agent. In one embodiment, the subject is receiving anti-viral and/or antibiotic treatment. In a more particular embodiment, the subject is receiving an anti-viral agent. In another embodiment, the subject has previous received an anti-viral agent. In a further embodiment, the additional therapeutic agent is an anti-viral agent. In even more particular embodiments, the anti-viral agent is selected from olsetamivir, remdesivir, ganciclovir, lopinavir, ritonavir and zanamivir. In another embodiment the anti-viral agent is selected from abacavir, stavudine, valganciclovir, cidofovir, entecavir, amivudine, maraviroc, azidothymidine, amprenavir, nelfinavir and dolutegravir. In a further embodiment the anti-viral agent is remdesivir.

In one embodiment, the subject is receiving oseltamivir (75 mg every 12 hours orally). In another embodiment, the subject is receiving ganciclovir (0.25 g every 12 hours intravenously). In another embodiment, the subject is receiving lopinavir/ritonavir (400/100 mg twice daily orally). In another embodiment, the subject is receiving remdesivir 200 mg intraveneously on Day 1 followed by 100 mg daily for 9 days. In another embodiment, the subject is receiving remdesivir 200 mg intraveneously on Day 1 followed by 100 mg daily for 4 days.

In some embodiments, the additional therapeutic agent is a steroid, corticosteroid or antimalarial agent.

In one embodiment the subject is receiving, has received or will receive a steroid. In another embodiment the subject is receiving a steroid. Thus, in a further embodiment, the additional therapeutic agent is a steroid. In one embodiment the steroid is selected from dexamethasone and methylprednisolone. In another embodiment the steroid is dexamethasone. In another embodiment the steroid is methylprednisolone. In one embodiment dexamethasone is dosed at from 0.1 to 0.2 mg/Kg. In another embodiment methylprednisolone is dosed at from 0.5 to 1 mg/Kg.

In one embodiment the subject is receiving, has received or will receive convalescent plasma. Thus, in a further embodiment the additional therapeutic agent is convalescent plasma. In another embodiment, the subject will receive convalescent plasma at least 48 hours before administration of the BlyS antagonist. In a further embodiment, the subject has received convalescent plasma about 12 weeks or more than 12 weeks prior to BlyS antagonist treatment.

In one embodiment the subject is receiving, has received or will receive high dose corticosteroids (HDCS) and broad-spectrum immunosuppressive agents. Thus, in a further embodiment, the additional therapeutic agent is high dose corticosteroids and a broad-spectrum immunosuppressant agent. First line standard therapies include cyclophosphamide (CYC) and HDCS for induction followed by azathioprine (AZA) for maintenance, or mycophenolate mofetil (MMF) and HDCS for induction followed by MMF for maintenance.

In one embodiment, the BlyS antagonist, such as the anti-BlyS antibody (e.g. belimumab) is co-administered with High Dose Corticosteroids (HDCS) and Cyclophosphamide (CYC) for induction therapy followed by Azathioprine (AZA) for maintenance therapy; or HDCS and Mycophenolate Mofetil (MMF) for induction therapy followed by MMF for maintenance therapy. In a further embodiment, the induction therapy is started within 60 days of the first dose of the anti-BlyS antibody.

In another embodiment, the anti-BlyS antibody may be combined with other biologics or therapeutics such as other antibodies or therapies such as anti-CD20 antibodies such as for example rituximab as described hereinbefore.

In one aspect the invention is described according to the following numbered paragraphs.
1. A BlyS antagonist for use in the treatment of an autoimmune condition induced following a viral infection.
2. The BlyS antagonist for use according to paragraph 1, wherein the autoimmune condition is chronic, preferably wherein the condition is chronic fatigue syndrome, myalgic encephalomyelitis (ME) and/or Long Covid, more preferably wherein the condition is Long Covid.
3. The BlyS antagonist for use according to paragraph 1 or paragraph 2, wherein the viral infection is an enteric virus infection, a herpes virus infection, an influenza infection or a coronavirus infection.
4. The BlyS antagonist for use according to paragraph 3, wherein the enteric virus infection is a Coxsackie B virus (CVB) or rotavirus infection, wherein the influenza virus infection is an influenza A infection, or wherein the coronavirus infection is a SARS-COV-2 infection.
5. The BlyS antagonist for use according to paragraphs 1 to 4, wherein the autoimmune condition is characterised by the presence of autoantibodies in a subject.
6. The BlyS antagonist for use according to paragraph 5, wherein the autoantibodies are present in the blood of the subject.
7. The BlyS antagonist for use according to paragraph 5 or paragraph 6, wherein the autoantibodies are selected from: anti-nuclear antibodies (ANAs), anti-rheumatoid factor (RF) antibodies, anti-double-stranded DNA (dsDNA) antibodies, anti-extra nuclear antigens (ENA) antibodies, anti-ribosomal-P antibodies, anti-RNP-70 antibodies, anti-Sjögren's-syndrome-related antigen A (SS-A) and/or anti-Sjögren's-syndrome-related antigen B (SS-B) antibodies, anti-Sm antibodies, anti-phospholipid antibodies, anti-anterior cruciate ligament (ACL) antibodies, anti-lupus anticoagulant (LAC) antibodies, and/or anti-beta-2-glycoprotein-1 antibodies.
8. The BlyS antagonist for use according to paragraph 7, wherein the titre and/or levels of anti-nuclear antibodies (ANAs) and/or anti-rheumatoid factor (RF) antibodies are those seen in systemic lupus erythematosus (SLE).
9. The BlyS antagonist for use according to paragraph 7 or paragraph 8, wherein the titre of anti-nuclear antibodies (ANAs) is greater than 1:80 and/or anti-rheumatoid factor (RF) antibodies is greater than 20 IU/mL.
10. The BlyS antagonist for use according to paragraphs 1 to 9, wherein the autoimmune condition is characterised by a serum ferritin level above 150 ng/ml in females and above 300 ng/mL in males in a subject.
11. The BlyS antagonist for use according to paragraphs 1 to 10, wherein the autoimmune condition is characterised by a C-reactive protein level above 10 mg/L in a subject.
12. The BlyS antagonist for use according to paragraphs 1 to 11, wherein the BlyS antagonist is an anti-BlyS antibody.
13. The anti-BlyS antibody for use according to paragraph 12, wherein the antibody is belimumab.
14. The anti-BLys antibody for use according to paragraph 12 wherein the antibody comprises CDRH1 of SEQ ID NO: 1; CDRH2 of SEQ ID NO: 2; CDRH3 of SEQ ID NO: 3; CDRL1 of SEQ ID NO: 4; CDRL2 of SEQ ID NO: 5 and CDRL3 of SEQ ID NO: 6.
15. The anti-BLyS antibody for use according to paragraph 14, wherein the antibody comprises a variable heavy chain sequence of SEQ ID NO: 7 and a light chain variable sequence of SEQ ID NO: 8.
16. The anti-BLys antibody for use according to paragraph 15, wherein the antibody comprises a heavy chain sequence of SEQ ID NO: 9 and a light chain sequence of SEQ ID NO: 10.
17. The anti-BlyS antibody for use according to paragraphs 12 to 16, wherein the antibody is administered intravenously (IV).
18. The anti-BlyS antibody for use according to paragraph 17, wherein the antibody is administered to a subject at a dose of 10 mg/kg.
19. The anti-BlyS antibody for use according to paragraph 18, wherein the antibody is administered every 2 weeks.
20. The anti-BLys antibody for use according to paragraphs 1 to 16, wherein the antibody is administered subcutaneously.
21. The anti-BLys antibody for use according to paragraph 20, wherein the antibody is administered to a subject at a unit dose of 200 mg a week.
22. The anti-BLys antibody for use of to paragraph 20, wherein the antibody is administered to a subject at a unit dose of 400 mg a week.
23. The anti-BLys antibody for use according to paragraph 21 or paragraph 22, wherein the antibody is administered at a dose of 400 mg a week for at least 4 weeks then at a dose of 200 mg once weekly thereafter.
24. The anti-BLys antibody for use according to paragraphs 1 to 23, wherein the antibody is administered intravenously prior to subcutaneous administration.
25. The anti-BLys antibody for use according to paragraph 24, wherein the antibody is administered intravenously at a loading dose of 10 mg/kg for at least 1 week prior to subcutaneous administration.
26. The BlyS antagonist for use according to paragraphs 1 to 25, wherein the treatment further comprises administration of an additional therapeutic agent.
27. The BlyS antagonist for use according to paragraph 26, wherein the additional therapeutic agent is an anti-viral and/or antibiotic agent.
28. The BlyS antagonist for use according to paragraph 26 or paragraph 27, wherein the additional therapeutic agent is a steroid, corticosteroid or antimalarial agent.
29. An anti-BlyS antibody for use in the treatment of Long Covid, wherein the anti-BlyS antibody is belimumab and/or is as defined in any one according to paragraphs 14 to 16.
30. Use of a BlyS antagonist in the manufacture of a medicament for the treatment of an autoimmune condition induced following a virus infection.
31. The use according to paragraph 30, wherein the autoimmune condition is chronic, preferably wherein the condition is chronic fatigue syndrome, myalgic encephalomyelitis (ME) and/or Long Covid, more preferably wherein the condition is Long Covid.
32. The use according to paragraph 30 or paragraph 31, wherein the viral infection is an enteric virus infection, a herpes virus infection, an influenza infection or a coronavirus infection.
33. The use according to paragraph 32, wherein the enteric virus infection is a Coxsackie B virus (CVB) or rotavirus infection, wherein the influenza virus infection is an influenza A infection, or wherein the coronavirus infection is a SARS-COV-2 infection.
34. The use according to paragraphs 30 to 33, wherein the autoimmune condition is characterised as defined in any one according to paragraphs 5 to 11.
35. The use according to paragraphs 30 to 34, wherein the BlyS antagonist is an anti-BlyS antibody.
36. The use according to paragraph 35, wherein the anti-BlyS antibody is an antibody as defined in any one according to paragraphs 13 to 16.
37. The use according to paragraph 35 or paragraph 36, wherein the medicament is administered as defined in any one according to paragraphs 17 to 25.
38. The use according to paragraphs 30 to 37, wherein the medicament additionally comprises a further therapeutic agent.
39. The use according to paragraph 38, wherein the further therapeutic agent is an anti-viral and/or antibiotic agent.
40. The use according to paragraph 38 or paragraph 39, wherein the further therapeutic agent is a steroid, corticosteroid or antimalarial agent.
41. A pharmaceutical composition for use in the treatment of an autoimmune condition induced following a viral infection, said pharmaceutical composition comprising a BlyS antagonist.
42. The pharmaceutical composition for use according to paragraph 41, wherein the autoimmune condition is chronic, preferably wherein the condition is chronic fatigue syndrome, myalgic encephalomyelitis (ME) and/or Long Covid, more preferably wherein the condition is Long Covid.
43. The pharmaceutical composition for use according to paragraph 41 or paragraph 42, wherein the viral infection is an enteric virus infection, a herpes virus infection, an influenza infection or a coronavirus infection.
44. The pharmaceutical composition for use according to paragraph 43, wherein the enteric virus infection is a Coxsackie B virus (CVB) or rotavirus infection, wherein the influenza virus infection is an influenza A infection, or wherein the coronavirus infection is a SARS-COV-2 infection.
45. The pharmaceutical composition for use according to paragraphs 41 to 44, wherein the autoimmune condition is characterised as defined in any one according to paragraphs 5 to 11.
46. The pharmaceutical composition for use according to paragraphs 41 to 45, wherein the BlyS antagonist is an anti-BlyS antibody.
47. The pharmaceutical composition for use according to paragraph 46, wherein the anti-BlyS antibody is an antibody as defined in any one according to paragraphs 13 to 16.
48. The pharmaceutical composition for use according to paragraph 46 or paragraph 47, wherein the pharmaceutical composition is administered as defined in any one according to paragraphs 17 to 25.
49. The pharmaceutical composition for use according to paragraphs 41 to 48, wherein the pharmaceutical composition additionally comprises a further therapeutic agent.
50. The pharmaceutical composition for use according to paragraph 49, wherein the further therapeutic agent is an anti-viral and/or antibiotic agent.
51. The pharmaceutical composition for use according to paragraph 49 or paragraph 50, wherein the further therapeutic agent is a steroid, corticosteroid or antimalarial agent.
52. A method for the treatment of an autoimmune condition induced following a viral infection in a subject in need thereof, said method comprising administering to said subject a therapeutically effective amount of a BlyS antagonist.
53. The method according to paragraph 52, wherein the autoimmune condition is chronic, preferably wherein the condition is chronic fatigue syndrome, myalgic encephalomyelitis (ME) and/or Long Covid, more preferably wherein the condition is Long Covid.
54. The method according to paragraph 52 or paragraph 53, wherein the viral infection is an enteric virus infection, a herpes virus infection, an influenza infection or a coronavirus infection.
55. The method according to paragraph 54, wherein the enteric virus infection is a Coxsackie B virus (CVB) or rotavirus infection, wherein the influenza virus infection is an influenza A infection, or wherein the coronavirus infection is a SARS-COV-2 infection.
56. The method according to paragraphs 52 to 55, wherein the the autoimmune condition is characterised as defined in any one according to paragraphs 5 to 11.
57. The method according to paragraphs 52 to 56, wherein the BlyS antagonist is an anti-BlyS antibody.
58. The method according to paragraph 57, wherein the anti-BlyS antibody is an antibody as defined in any one according to paragraphs 13 to 16.
59. The method according to paragraph 57 or paragraph 58, wherein the anti-BlyS antibody is administered as defined in any one according to paragraphs 17 to 25.
60. The method according to paragraphs 52 to 59, wherein the method comprises administering the medicament as defined in any one according to paragraphs 30 to 40 or the pharmaceutical composition as defined in any one according to paragraphs 41 to 51.
61. The method according to paragraphs 51 to 60, wherein the method additionally comprises administration of a further therapeutic agent.
62. The method according to paragraph 61, wherein the further therapeutic agent is an anti-viral and/or antibiotic agent.
63. The method according to paragraph 61 or paragraph 62, wherein the further therapeutic agent is a steroid, corticosteroid or antimalarial agent.
64. A method for the treatment of Long Covid in a subject in need thereof, said method comprising administering to said subject a therapeutically effective amount of an anti-BlyS antibody, wherein the anti-BlyS antibody is belimumab and/or is as defined in any one according to paragraphs 14 to 16.
65. The BlyS antagonist for use according to paragraphs 1 to 28, the anti-BlyS antibody according to paragraph 29 or the method of any one according to paragraphs 52 to 64 wherein the subject is a human.

### EXAMPLES

### Example 1—Planned Studies

Planned studies to identify protein biomarkers in the plasma of patients infected with SARS-CoV-2 with particular focus on the B cell stimulation factor, BLyS are disclosed. These studies will be carried out in parallel with ongoing autoreactivity studies with the goal of identifying proteomic signatures correlated with loss of B cell tolerance and the presence of severe and lingering symptoms in COVID-19, such as in Long Covid. The results of this work will further support the use of Blys antagonists particularly belimumab in the treatment of Long Covid and other virally induced autoimmune conditions.

This will be investigated using two independent patient cohorts.

The first planned cohort, or ‘retrospective’ cohort, will include patients that have been highly characterized in previous studies in COVID-19 including high-dimensionality flow cytometry, SARS-COV-2-specific serology, and in some cases, single cell analysis. Specific samples of high interest will include longitudinal time points to identify persistent signatures in the blood following patient recovery.

The second planned cohort, or ‘recovery’ cohort, will include patients recruited from COVID-19 recovery clinics exhibiting ongoing symptoms, and that have been pre-screened for the presence of autoantibodies. We will identify an autoreactive cohort within these COVID-19 recovered patients and perform longitudinal testing to identify protein signatures of autoreactivity following COVID-19 recovery.

Cohort 1—Retrospective Cohort (n=130):
1. Pre-COVID Healthy Donors (HD) populations (demographically mixed) (n=10 patients);
2. Mild/Moderate COVID patients (acute collections) (n=20);
3. Severe/Critical COVID patients:
   a. Dexamethasone positive (n=20),
   b. Dexamethasone negative (n=20);
4. Active/flaring lupus patients (n=10);
5. Longitudinal sample collections tied to acute samples above (n=30); and
6. Multisystem Inflammatory Syndrome in Children (MIS-C) patients (n=20).
   Cohort 2—Recovery Cohort (Up to n=210):
1. 50 COVID-recovered patients with positive autoreactivity testing:
   a. Collection on presentation,
   b. 6 month follow up, and
   c. 1 year follow up;
2. 20 COVID-recovered patients with negative autoreactivity testing:
   a. Collection on presentation,
   b. 6 month follow up, and
   c. 1 year follow up.

Phase one will include high-throughput (1536-target) proteomics analysis of samples from cohort 1. Results will be collected and analyzed in the context of paired datasets including patient metadata, flow cytometry, and autoreactivity testing.

Phase two will be executed on completion of an interim analysis of phase one. Patients from cohort 2 will be pre-screened in real time for autoreactivity, with frozen samples banked for future study. They will be stratified into those displaying positive autoreactivity, and those that do not. Based on the results from the phase one analysis, collected cohort 2 samples will then have either undergo high-throughput (1536-target), or more directed targeting (96-target) analysis pipelines. If a more targeted approach is deemed more appropriate, the cohort size may be expanded.

The analysis will include detailed assessment of the proteomics results coupled with available flow cytometry characterization, disease outcomes, patient metadata, autoreactivity testing, disease characterization, and inflammatory biomarkers where available (from ICU patient testing), SARS-COV-2-specific serology, and autoantibody levels. Limited additional data obtained through single-cell RNA sequencing of B cell populations may be available through single cell transcriptomics analysis, and where available, will be included.

An optional phase three may be conducted subsequently or in parallel to phase two wherein samples from cohort 2 will be collected on several occasions on subsequent visits to the clinic/sample collection site. Such sample collection will provide insight as to the autoreactive protein signatures over a prolonged period following COVID-19 recovery.

### Example 2—Proteomics Study

190 human adult plasma samples obtained from a variety of disease states (described below) related to COVID-19 were submitted for blood proteomics assessment. Although initially a two-phase study was anticipated, a single analysis of all available samples were executed in a single run to investigate all protein markers available through the Olink Explore 3072 platform (3072 different protein markers were assessed consisting of 768 Inflammatory markers, 768 Cardiometabolic markers, 768 Neurologic markers and 768 Oncologic markers) for the following sample groups:
HD: Pre-pandemic healthy donors (n=9)
SLE: Lupus patients experiencing high disease activity (n=9)
Mild/Moderate: acute COVID-19 patients not requiring hospitalization (n=15)
ICU: acute COVID-19 patients requiring Intensive Care Unit (ICU) care (n=30)
CR: Patients recovered from COVID-19 with no lingering symptoms (n=28)
PASC: (Long covid and/or PASC) Patients recovered from COVID-19 with ongoing symptoms (n=99)

Ongoing scientific investigation of PASC has not revealed a clear cutoff point for the emergence of symptoms and instead reflects a continuum of disease beginning at the early phases of recovery. To this end, while the majority of patients within the PASC cohort meet the 12 week recovery period following infection criteria (61%), a significant fraction (39%) also reflects the important biological leadup period prior to formal diagnosis.

### Example 3—Differential Protein Expression

The datasets resulting from the full proteomics analysis were evaluated for quality control purposes and, with the exception of some evidence of sample interference in the acute-phase disease plasma (groups Mild/Moderate and ICU) in a subset of protein abundance markers, the overall dataset was deemed reliable and well-controlled. In general, normalized protein expression (NPX) distributions were appropriately centered and spread yielding high confidence in the use of the proteomics data to identify differential protein abundance in patients with uncomplicated recovery (CR) versus those suffering from ongoing symptoms consistent with PASC.

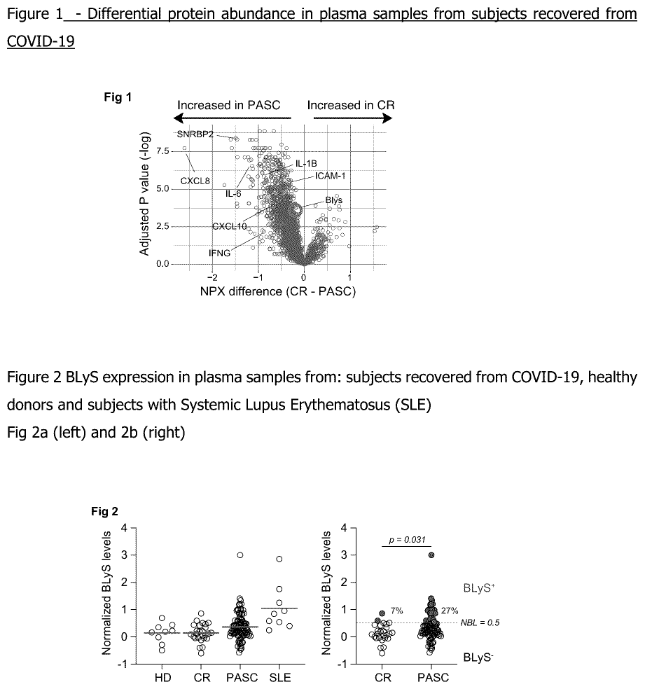

An assessment of differential NPX revealed more than 600 proteins with significant abundance differences between the groups (FIG. 1). While protein identities were diverse, many of the proteins identified were suggestive of an ongoing inflammatory process in PASC previously associated with disease severity in the acute phase of infection including IL-6 and CXCL10.

These observations of inflammatory marker expression were validated via KEGG pathway analysis of differentially abundant proteins with highly significant enrichment in cytokine-cytokine receptor interactions (p=4.97×10⁻²⁰), complement and coagulation cascades (p=8.50×10⁻¹²), apoptosis signaling (3.60×10⁻¹⁰), and TNF signaling pathway (p=1.23×10⁻⁹) as the most highly enriched pathways in the PASC cohort.

Specific investigation of BLyS protein expression across the recovery cohorts suggested increased levels in a subset of PASC patients (FIG. 2a). Importantly, patients in the CR group displayed similar levels of BLyS in the plasma as pre-pandemic healthy donors, while in contrast, some patients with PASC displayed elevated levels similar to SLE patients with high disease activity (FIG. 2a). Direct comparison of BLyS levels in the CR and PASC cohorts revealed significantly increased levels in the PASC cohort, (p-value<0.05 for example a p-value of 0.031) but suggested a PASC cohort heterogeneous for BLyS expression, with 27% of PASC patients displaying BLyS levels greater than 0.5 NPX (Log 2 scale) (FIG. 2b). Importantly, BLyS expression levels across the recovery cohorts significantly correlated with both markers of severe acute COVID-19, such as CXCL10, as well as recently identified markers indicating the emergence of PASC including Pentraxin 3 (PTX3) (FIG. 3).

### Example 4—B Cell Responses in PASC

Previously, work in identifying immune responses associated with severe COVID-19 disease outcomes have identified a non-canonical immune activation pathway, the extrafollicular (EF) B cell pathway, as a strong correlate of critical illness. While primary data in humans is still emerging, this pathways appears to be highly responsive to high-inflammation environments such as the one resulting from severe COVID-19. By bypassing traditional mechanisms of immune system selection, the EF pathway is capable of generating a robust antibody response against foreign proteins within just days of infection. However, through previous studies of autoimmune disease, this pathway is also associated with the emergence of new autoreactivity and peripheral tissue inflammation. As BLYS is elevated in many of these patients, and BLyS neutralization has previously been shown to be efficacious in controlling B-cell mediated autoreactive disease, it was important to understand if similar EF activation could be identified in PASC patients displaying high BLyS levels (for example on a Log 2 scale, 0.5×normalized protein expression levels compared with healthy controls).

Utilizing high-dimensional flow cytometry, the B cell compartments of 40 PASC patients (29 BLyS^{NEGATIVE} and 11 BLyS^{POSITIVE}) were assessed for the presence of differing B cell subtypes indicative of EF pathway activity. Interestingly, while previous studies in acute COVID-19 have identified significant expansion of antibody secreting cells correlating with severe disease, both CR and PASC patients showed low levels of these circulating cells similar to previous observations in HD populations (FIG. 4a). However, intermediates of the EF pathway upstream of antibody secreting cell differentiation, activated naive (aN) and double negative 2 (DN2) B cells, both displayed increased trends in PASC. An important metric in the assessment of EF B cell activity is the ratio of EF B cell responders versus more traditional germinal center-derived subsets. Assessment of this metric in PASC again showed a trend towards increased EF response in BLyS^{POSITIVE} patients, this time approaching significance (p=0.058). In all, these data are suggestive that while the EF pathway is not as highly active as the responses identified in active SLE and severe COVID-19 to date, it is nonetheless emphasized in PASC patients displaying higher levels of BLyS.

### Example 5—Autoreactivity in the PASC Cohort

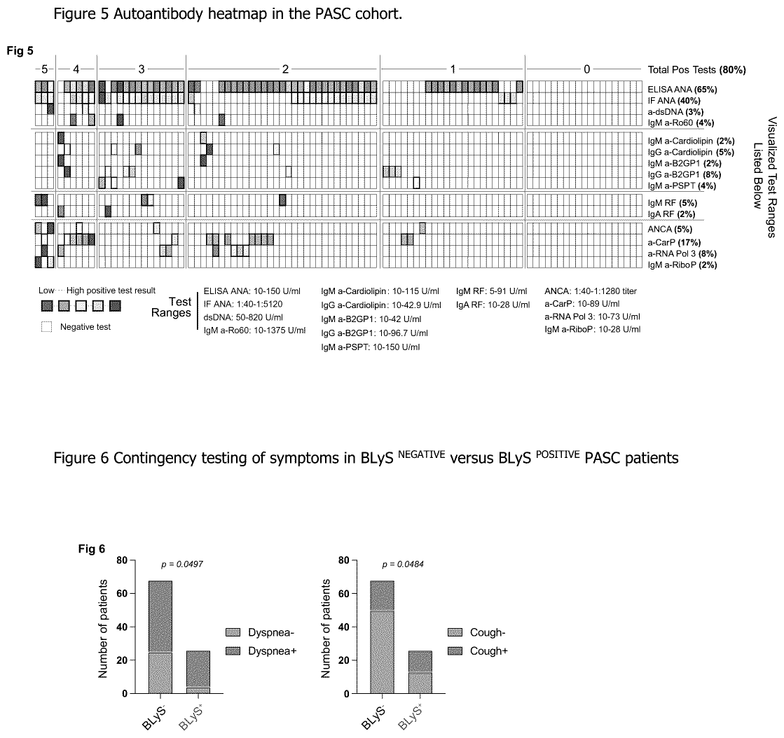

To identify relationships between PASC and autoantibody levels, plasma samples from PASC patients were submitted for broad clinical autoantibody testing through the Exagen Inc. AVISE pathology platform. This platform makes use of FDA-approved clinical tests targeting a broad array of connective tissue disorders including SLE, rheumatoid arthritis, vasculitis, etc. In total 31 antigens were tested, with positive test results displayed in FIG. 5. Similar to the high levels of autoreactivity identified in severe COVID-19, 80% of the patients tested in the screen displayed at least 1 positive clinical test. These autoantobody reactivities were not random, with most reactivity identified against anti-nuclear antigens, carbamylated proteins, and phospholipids. Of interest, patients with high BLyS levels were more likely to display 3 or more reactivities than their low-BLyS counterparts (Table 1), and two reactivities, rheumatoid factor (RF) and Antineutrophil Cytoplasmic Antibodies (ANCA) were identified exclusively in the BLyS^{POSITIVE} group.

TABLE 1
Frequency of total autoreactive positive tests
in BLyS^{POSITIVE} versus BLyS^{NEGATIVE} PASC patients
in Exagen Inc. AVISE screening platform

| Positive Tests | 0 | 1 | 2 | 3+ |
|---|---|---|---|---|
| BLyS− | 25.4% | 40.8% | 32.4% | 1.4% |
| BLyS+ | 17.9% | 42.9% | 21.4% | 17.9% |

As mentioned above, the emergence of EF response pathways in SLE is responsible for at least a subset of patients experiencing high disease activity. Additionally, newer studies have identified similar mechanisms in COVID-19—solidifying naive-derived EF responses as a source of emerging autoreactive responses. With indications of trending EF activation in PASC it is important to understand both if autoreactivity is associated with persistence of symptoms, and if those symptoms can be alleviated through the reduction of self-targeted antibody responses as has been previously shown in other BLyS-targeted therapy.

### Example 6—Symptom Heterogeneity in the PASC Cohort

Due to an indication that BLYS may correlate with a distinct subset of PASC patients, it was important to understand if patients with elevated BLyS levels presented with specific clinical symptoms. To this end, patient intake forms detailing ongoing PASC symptoms and collected at the time of blood collection upon presentation in COVID-19 recovery clinics were carefully reviewed to document potential associations with BLyS status. Interestingly, of 15 common symptoms reported in more than 10% of the overall PASC patient cohort, 7 showed increased associations with the BLyS^{POSITIVE} patient subset with at least 10% increased frequency versus BLyS^{NEGATIVE} counterparts (Table 2).

TABLE 2
Frequency of PASC patients exhibiting indicated symptoms
in BLyS^{POSITIVE} versus BLyS^{NEGATIVE} subgroups.

| Symptom | Dyspnea | Fatigue | Weakness | Cough | Dizzy |
|---|---|---|---|---|---|
| BLyS− | 68.7% | 59.7% | 17.9% | 28.4% | 31.3% |
| BLyS+ | 80.0% | 75.0% | 30.0% | 55.0% | 15.0% |
| | Headache | Joint pain | Chest pain | Brainfog | Anosmia |
| BLyS− | 26.9% | 13.4% | 19.4% | 47.8% | 11.9% |
| BLyS+ | 35.0% | 25.0% | 35.0% | 45.0% | 20.0% |
| | Dysgeusia | Myalgia | Sleep | Anxiety | Depression |
| BLyS− | 21.1% | 21.1% | 14.0% | 17.5% | 17.5% |
| BLyS+ | 16.7% | 16.7% | 11.1% | 22.2% | 27.8% |

Specific symptoms showed particular emphasis, with both dyspnea and cough identified as significantly more prevalent in BLyS^{POSITIVE} patients through contingency testing (FIG. 6).

### Example 7—Analysis

Overall, broad proteomics analysis of patients with ongoing sequelae of COVID-19 is consistent with an ongoing inflammatory event persisting well into the recovery phase of disease. Targeted analysis of BLYS levels indicate a positive association with inflammatory markers implicated in both the acute and recovery phase of disease and show trends in the persistence of the EF response pathway known to correlate with disease activity in autoimmune disease. Broad autoreactivity across the cohort, and specific reactivities within the BLyS^{POSITIVE} cohort, suggest potential implications of autoreactivity in ongoing symptom presentation. Identification of specific symptoms with significant associations in the BLyS^{POSITIVE} subpopulation further suggests that BLyS levels may be elevated in a specific subset of PASC patients displaying autoimmune-like tendency.

1. A BlyS antagonist for use in the treatment of Long Covid and/or post-acute sequelae SARS-COV-2 infection (PASC).

2. The Blys antagonist for use in the treatment of Long Covid and/or post-acute sequelae SARS-COV-2 infection (PASC) which results from infection with the human coronavirus SARS-COV-2.

3. The BlyS antagonist for use according claim 1, wherein the BlyS antagonist is an anti-BlyS antibody.

4. The anti-BlyS antibody for use according to claim 3, wherein the anti-BlyS antibody is belimumab or a variant thereof.

5. The anti-BlyS antibody for use according to claim 4, wherein the variant antibody binds to the same epitope as belimumab.

6. A pharmaceutical composition comprising a BlyS antagonist for use according to claim 1.

7. The pharmaceutical composition of claim 6, wherein the composition is administered to a patient in need thereof at least 4 weeks or at least 8 weeks or at least 12 weeks after the initial viral infection.

8. The pharmaceutical composition of claim 6, wherein the pharmaceutical composition is administered to a patient diagnosed as suffering from dyspnea and/or cough.

9. The pharmaceutical composition of claim 6, wherein the pharmaceutical composition is administered to a patient diagnosed as having at least 2 subsets of autoantibodies in their blood.

10. The pharmaceutical composition of claim 6, wherein the pharmaceutical composition is administered to a patient that tests positive for rheumatoid factor (RF) and/or Anti neutrophil Cytoplasmic Antibodies (ANCA).

11. The pharmaceutical composition of claim 6, wherein the pharmaceutical composition is administered to a patient that tests positive for anti nuclear antibodies (ANA) and/or anti-phospholipid antibodies.

12. The pharmaceutical composition of claim 6, wherein the pharmaceutical composition is administered to a patient diagnosed as having one or more of Blys, IFN-β, PTX3, IFN-lambda 2/3, and/or IL-6 in their blood.

13. A method of treating Long Covid and/or post-acute sequelae SARS-COV-2 infection (PASC) in a human comprising the steps of:
i) testing for levels of serum cytokines, Blys, IFN-β, PTX3, IFN-lambda 2/3, and/or IL-6;
ii) comparing/determining the levels of any of the results of step i) with a healthy reference level; and
iii) if the levels are at least 2 fold higher than the healthy reference level, then administering a therapeutically effective amount of a Blys antagonist.

14. The method of claim 13, further including obtaining a sample from said human.


SEQUENCE LISTING (10 sequences)

SEQ ID NO: 1 - protein, 10 aa, Artificial
  belimumab CDRH1
GGTFNNNAIN

SEQ ID NO: 2 - protein, 17 aa, Artificial
  belimumab CDRH2
GIIPMFGTAKYSQNFQG

SEQ ID NO: 3 - protein, 14 aa, Artificial
  belimumab CDRH3
SRDLLLFPHHALSP

SEQ ID NO: 4 - protein, 11 aa, Artificial
  belimumab CDRL1
QGDSLRSYYAS

SEQ ID NO: 5 - protein, 7 aa, Artificial
  belimumab CDRL2
GKNNRPS

SEQ ID NO: 6 - protein, 11 aa, Artificial
  belimumab CDRL3
SSRDSSGNHWV

SEQ ID NO: 7 - protein, 123 aa, Artificial
  belimumab VH
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSS

SEQ ID NO: 8 - protein, 109 aa, Artificial
  belimumab VL
SSELTQDPAVSVALGQTVRVTCQGDSLRSYYASWYQQKPGQAPVLVIYGKNNRPSGIPDRFSGSSSGNTASLTITGAQAEDEADYYCSSRDSSGNHWVFGGGTELTVLG

SEQ ID NO: 9 - protein, 453 aa, Artificial
  belimumab heavy chain
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial
  belimumab light chain
SSELTQDPAVSVALGQTVRVTCQGDSLRSYYASWYQQKPGQAPVLVIYGKNNRPSGIPDRFSGSSSGNTASLTITGAQAEDEADYYCSSRDSSGNHWVFGGGTELTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS